(19) United States
(12) Patent Application Publication
(10) Pub. No.: US 2014/0205272 A1
(43) Pub. Date: Jul. 24, 2014

(54) CONDUCTIVE COMPOSITE WICK AND METHOD OF MAKING AND USING THE SAME

(71) Applicants: William Graham Midgette, Grayson, GA (US); Guoqiang Mao, Peachtree City, GA (US); Timothy Martin, Newnan, GA (US); Edward Jino Kim, Riverdale, GA (US); Christopher Lynch, Atlanta, GA (US)

(72) Inventors: William Graham Midgette, Grayson, GA (US); Guoqiang Mao, Peachtree City, GA (US); Timothy Martin, Newnan, GA (US); Edward Jino Kim, Riverdale, GA (US); Christopher Lynch, Atlanta, GA (US)

(73) Assignee: POREX CORPORATION, Fairburn, GA (US)

(21) Appl. No.: 14/238,902

(22) PCT Filed: Aug. 13, 2012
(86) PCT No.: PCT/US2012/050530

§ 371 Date: Feb. 14, 2014

Related U.S. Application Data
(60) Provisional application No. 61/523,439, filed on Aug. 15, 2011.
(60) Provisional application No. 61/547,797, filed on Oct. 17, 2011.

(51) Int. Cl. A61L 9/03; A01M 1/20

(52) U.S. Cl. CPC A61L 9/037; A01M 1/2077
USPC 392/395

(57) ABSTRACT

This invention relates to a conductive composite wick comprising a porous wicking element coupled a conductive element for releasing vaporizable materials from devices such as an air freshener device or insect control system.

### PRIOR RELATED APPLICATIONS

The present application claims the benefit of priority to U.S. Provisional Application No. 61/523,439 filed Aug. 15, 2011 and to U.S. Provisional Application No. 61/547,797 filed Oct. 17, 2011, each of which is incorporated herein by reference in its entirety.

### FIELD OF THE INVENTION

This invention relates to conductive composite wicks comprising a porous wicking element and a conductive element for releasing vaporizable materials into the atmosphere from devices, such as an air freshener device or insect control system.

### BACKGROUND

Many people place air fresheners in a room to cover up odors in the room or to add a fragrant scent to the air. The need for effectively combating airborne malodors in homes and enclosed public buildings, by odor masking or destruction, is well established, as is the dispensing of insect control materials for killing or deterring insects. Various kinds of vapor-dispensing devices have been employed for these purposes. In particular, wicking devices are well known for dispensing vaporizable materials, such as a fragrance, deodorant, disinfectant, insecticide or insect repellant, into the atmosphere. A typical wicking device utilizes a combination of a wick and an emanating region to dispense a volatile liquid from a liquid reservoir.

Many air fresheners are commercially available. Air fresheners that utilize wicking action and/or are plug-in diffusers are particularly popular with consumers. Reed diffusers became popular because they do not require power, are low in cost and can be designed into different shapes and colors to match and decorate the environment. Plug-in diffusers are also known in the art. In these devices, a resistance heater is disposed in a housing, from which electrical prongs extend directly. When the prongs are plugged into a wall socket, the resistance heater generates heat. A substance, such as a fragrance or an insect repellant, to be emitted into the air is maintained, typically in liquid form, in close proximity to the heater. As the heater heats the substance, controlled amounts are vaporized and emitted into the surrounding atmosphere. These devices are well suited for domestic use, especially in rooms such as kitchens and bathrooms, because they provide a continuous, controlled flow of a desired substance into the air.

The wick materials for those devices are fiber based plastic materials, sintered porous plastic materials or ceramic based materials. These materials are insulators and do not conduct the heat and electricity. When the devices need to be heated, such as the plug in devices, poor conductivity results in low fragrance delivery into the air and requires higher energy. Attempts have been made to improve the fragrance delivery rate such as using a circulating fan, increasing the temperature and applying a larger diameter wick. All these solutions increase the product cost. Increasing the temperature may require the use of more expensive ceramic based porous wicks. Large wicks result in a bulky product and uneven heating properties.

Wicks with metal insertion have been used in flame based applications. U.S. Pat. No. 6,444,156 discusses the disadvantage of using a metal core wick in gel candles. In this application the metal core provides the mechanical rigidity for the wick to withstand the pressure and maintain its location during the manufacturing process. U.S. Pat. No. 6,333,009 teaches use of a metal tube as a heating element for an oil burning lamp. However, this application is also flame based.

Accordingly, there is a need for a wick that has better conductivity and delivers more vaporizable materials under relatively low temperature. These types of wicks would require less heating than current non-conductive wicks. There is a need for wicks that have conductive components providing thermal and/or electrical conductivity and heating capability that provides users controlled delivery rates of vaporizable material without the use of flame or a fan. Additionally, there is a need for simple devices that provide multiple delivery capabilities using selectively activated thermally or electrically conductive circuits.

### SUMMARY

The present invention provides conductive composite wicks, devices and methods for wicking and evaporating a liquid in a container of vaporizable materials, such as an air freshener, a perfume, a disinfectant, an insect repellant or an insecticide. These conductive composite wicks comprise a porous liquid wicking element coupled to a conductive element for releasing vaporizable materials into the atmosphere from in devices such as an air freshener device or insect control system.

Porous wicking elements of the present invention include different types of open cell porous media. These porous wicking media include open cell foams, felts, felts bounded with thermosetting resins, woven fibers, porous media comprising thermosetting resins and inorganic fillers, extruded plastic hollow tubes, and porous media comprising synthetic and natural cellulose materials. In some embodiments, porous wicking elements comprise sintered porous plastic. In other embodiments, porous wicking elements comprise fibrous materials, such as monocomponent fibers or bicomponent fibers.

Conductive elements may be electrically conductive, thermally conductive or both electrically conductive and thermally conductive. In some embodiments, conductive elements comprise carbon. In other embodiments, conductive elements comprise a metal or metallic alloy. Conductive elements are coupled to a power source for electrical or thermal conductivity.

The conductive composite wicks of the present invention require less heating and provide more uniform heating than currently available non-conductive wicks. In some embodiments, these wicks have conductive element channels that can provide heating, sensing and controlling capability for the liquid delivery devices. The porous wicking element may be biodegradable. The porous wicking element may be hydrophilic. The porous wicking element may be biodegradable and hydrophilic.

The wicks of the present invention can be made from different materials and can be used for delivering a vaporizable material into an environment, such as a room environment. Vaporizable materials include aqueous based fragrances and in some embodiments are fragrance formulations that have a water composition over 50% by weight. The wicks of the present invention can also deliver non-aqueous based fragrances into an environment. The wicks of the present invention can also deliver other vaporizable materials into an environment.

The conductive composite wicks of the present invention are optionally treated to increase the surface energy of the porous wicking element and improve the wicking rate. One way to increase this surface energy is to treat porous wicking elements using plasma. This can be a batch process at low pressure or an inline process at or above atmospheric pressure. A number of gases can be energized to react with the surface of the fiber to create hydrophilic moieties and improve hydrophilicity. Gases include, but are not limited to, oxygen, air, nitrogen, argon and a combination thereof. Various exposure times, pressures, and energies are used during the plasma process depending on the desired product requirements. The conductive composite wicks of the present invention may be optionally activated by employing finishing agents for improved hydrophilicity. In one embodiment, finishing agents may be applied to the bicomponent fibers. In another embodiment, finishing agents are present in the commercially available bicomponent fibers.

The present invention also provides novel devices for delivering vaporizable materials to the atmosphere using the conductive composite wicks of the present invention.

Conductive composite wicks can provide feedback signals to the controlling device for providing more consistent vaporizable liquid delivery. These composite wicks can also alert a user. For example, a user can be alerted as to the level of liquid being delivered, which liquid is being delivered, or if the container is empty and requires addition of vaporizable material. In many cases, once a user initially places an air freshener in a room, he or she typically forgets about the amount of vaporizable air freshener in the container. After extended use, air fresheners often are empty for some time without being noticed. This may be attributed, in part, to the subtly of the gradual decline in scent as well as a person's olfactory scent adaptation. In one embodiment, the devices provide another sensory signal, for example an auditory or visual signal indicating that it is time for a new air freshener or to replace the vaporizable material in the reservoir of the air freshener.

Conductive composite wicks can also act as resistors that generate heat when power is applied to them. In this case, the conductive components in the composite wicks function as heating elements in the device and an external heating element in the housing is not needed. Composite wicks with electrical and/or thermal conductivity can also be part of a circuit that controls the delivery of vaporizable material, or controls a light or sound feature of the devices. For example the composite wick can have light features attached to it, such as light emitting diode (LED) or fiber optics. This embodiment would provide a light feature for decorative purposes during use of the device. Different light features might also indicate different liquid delivery rates or lack of vaporizable material in the reservoir.

In one embodiment, the duration of energy applied to the composite wick can affect the release profile of the vaporizable material. In another embodiment, the amount of energy applied to the conductive composite wick can affect the amount of the vaporizable material released. The conductive composite wicks can be used to provide specific release profiles of vaporizable material by modulating the frequency, duration and/or amplitude of energy applied to the conductive composite wick. In one embodiment, a timer circuit known to one of ordinary skill in the art can deliver power to the conductive composite wick at specific times and for specific durations. In another embodiment, modulating the electrical energy applied to the conductive composite wick can modulate the amount of vaporizable material released. Modulating the frequency of release of the vaporizable material can decrease sensory adaptation to the vaporizable material.

Conductive composite wicks described herein with electrical and thermal conductivity can have many different shapes, such as rods, sheet, webs, or another profile.

Another embodiment of this invention is the method of using a conductive composite wick to deliver the vaporizable material into the environment. In one embodiment, a portion of the conductive composite wick is immersed in the reservoir of vaporizable material and the conductive element is attached to a heating element that is powered by an alternating current (AC) electric source, a direct current source electric source or a direct current (DC) battery. In another embodiment, a portion of the conductive composite wick is immersed in the reservoir of vaporizable material and the conductive element is attached to a power source such as an alternating current (AC) electric source, a direct current source electric source or a direct current (DC) battery.

### BRIEF DESCRIPTION OF THE DRAWINGS

FIG. 1 is a perspective view of an embodiment of a conductive composite wick 10 according to the present invention. A metal rod 12 is embedded in a porous wicking media 14.

FIG. 2 is a perspective view of an embodiment of a conductive composite wick 20 according to the present invention. A hollow metal tube 22 is embedded in a porous wicking media 24.

FIG. 3 is a perspective view of an embodiment of a conductive composite wick 30 according to the present invention. A metal wire 32 wraps around the outer surface of a porous wicking media 34.

FIG. 4 is a schematic representation of: A) a longitudinal section of a flexible, hollow wick 42 comprised of porous wicking media; and, B) a longitudinal section of a hollow wick 44 comprised of porous wicking media.

FIG. 5 is a schematic representation of a container 50 containing a volatile liquid 56 containing fragrance, a heating element 58 with a wire 59 for connection to a power source, a metal rod 52, a metal tube 53 and a metal wire 54 connected to the heating element and extending through the volatile liquid and through the opening in the container.

FIG. 6 is a schematic representation of a container 60 containing a volatile liquid 66, a heating element 68 with a wire 69 for connection to a power supply, metal elements 63 connected to the heating element and extending through the volatile liquid and through the opening in the container, and hollow wicks 62 comprised of porous wicking media placed over the metal elements.

FIG. 7 is a schematic representation of: A) hollow flexible wicks 70 comprised of porous wicking media; B) a container 72 containing a volatile liquid 74 containing fragrance, a heating element 78 with a wire 79 for connection to a power supply, and metal elements 75 connected to the heating element and extending through the volatile liquid and through the opening in the container; and, C) and hollow wicks 70 placed over the metal elements.

FIG. 8 is a perspective view of an embodiment of a conductive composite wick 80. A carbon fiber tow 82 twists together with a porous fiber wicking media 84 comprising a bicomponent fiber sliver.

FIG. 9 is a cross sectional view 90 and a longitudinal section view 91 of another embodiment of a conductive composite wick. The porous fiber wicking media 94 contains two channels housing conductive carbon fibers 92.

FIG. 10 is a cross sectional view of another embodiment of a conductive composite wick in the form of a pultruded conductive composite wick sheet 100. The wick is in sheet form and the carbon fiber component 102 is sandwiched between porous wick layers 104.

FIG. 11 is a perspective view of another embodiment of a conductive composite wick 110. The carbon fibers 112 have an arrangement resembling a single ribbon or sheet in the composite wick and are surrounded by the porous wicking media 114.

FIG. 12 is a perspective view of another embodiment of a conductive composite wick 120. The carbon fibers 122 have an arrangement resembling a crossed ribbon or crossed sheets in the composite wick and are surrounded by the porous wicking media 124.

FIG. 13 is a perspective view of another embodiment of a conductive composite wick 130. The carbon fibers 132 are in two channels in a dual core arrangement in the composite wick and are surrounded by the porous wicking media 134.

FIG. 14 is a perspective view of yet another embodiment of a conductive composite wick 140. The carbon fibers 142 are in three channels in a triple core arrangement in the composite wick and are surrounded by the porous wicking media 144.

FIG. 15 is a perspective view of another embodiment of a conductive composite wick 150. The carbon fibers 152 are in a helical arrangement with the porous wicking media 154 on the surface of the composite wick.

FIG. 16 is a perspective view of still another embodiment of a conductive composite wick 160. The carbon fibers 162 are in an annular or donut shape surrounding a core 162 of porous wicking material and contained in an outer layer of porous wicking material 166 in the composite wick.

FIG. 17 is a cross sectional view of another embodiment of a conductive composite wick 170. The wick is in sheet form and the carbon fiber component 172 is sandwiched between porous wick layers 174. Various shapes may be die cut from the sheet such as the tree shape 176 shown in the figure.

FIG. 18 is a cross sectional and perspective view of another embodiment of a conductive composite wick 180. The wick is in an extruded shape resembling a star with the carbon fiber component 182 located at the core of the wick and contained in porous wicking material 184.

FIG. 19 is a schematic representation of a container 190 containing a vaporizable material in fluid 196 in the reservoir of the container, a heating element 198 with a wire 199 to connect to a power supply, two conductive composite wicks 191 with carbon fiber elements 193, contained within porous wicking material 192, connected to the heating element and extending through the fluid and through the opening in the container.

FIG. 20 is a schematic representation of a container 260 containing a volatile liquid 266, a heating element 268 with a wire 269 for connection to a power supply, conductive elements 263 connected to the heating element and extending through the volatile liquid and through the opening in the container, and hollow wicks 262 comprised of porous wicking media placed over the conductive elements.

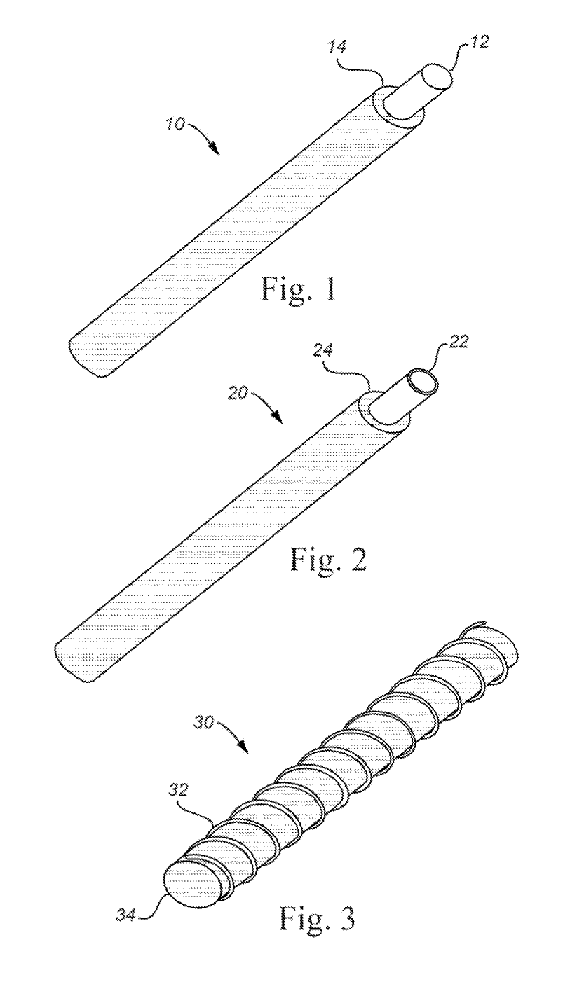

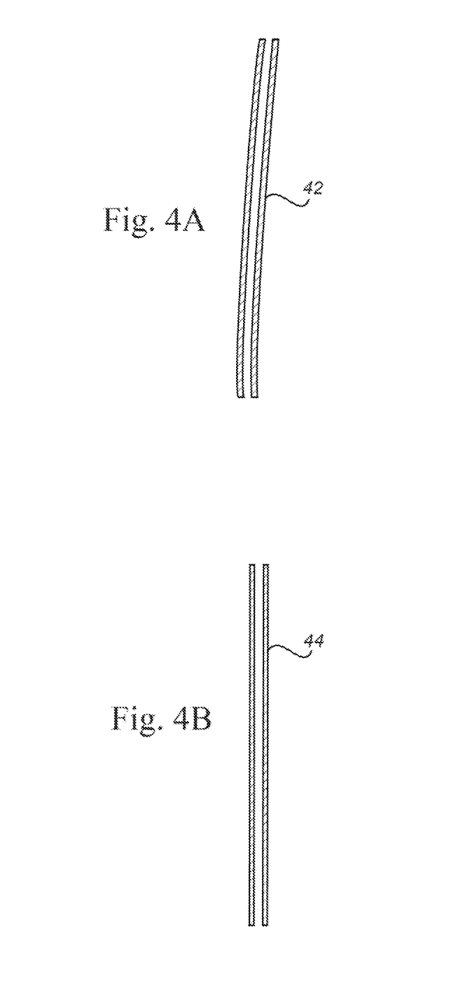

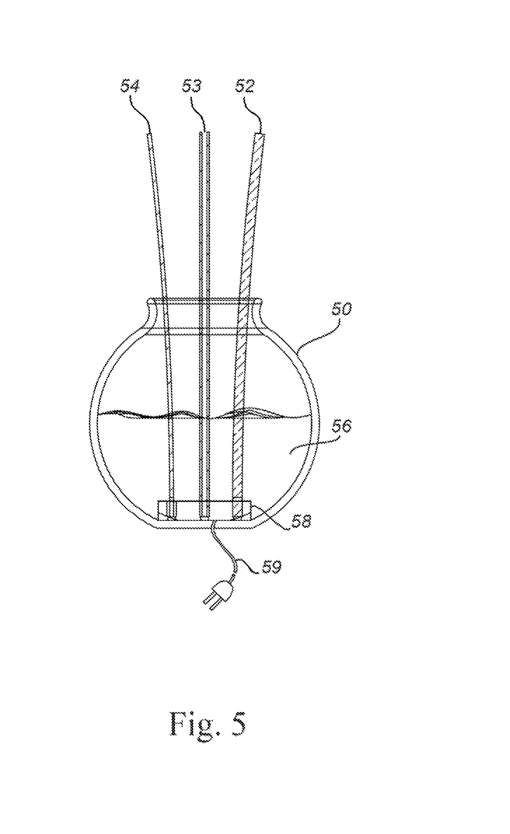

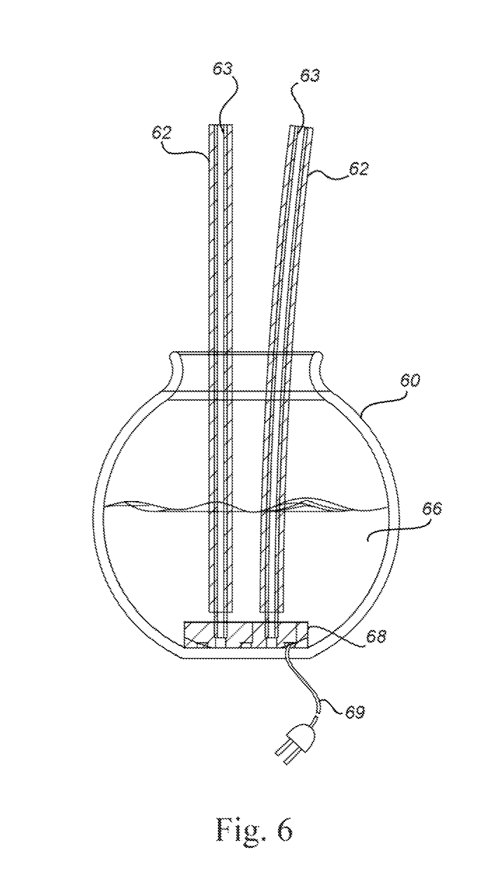

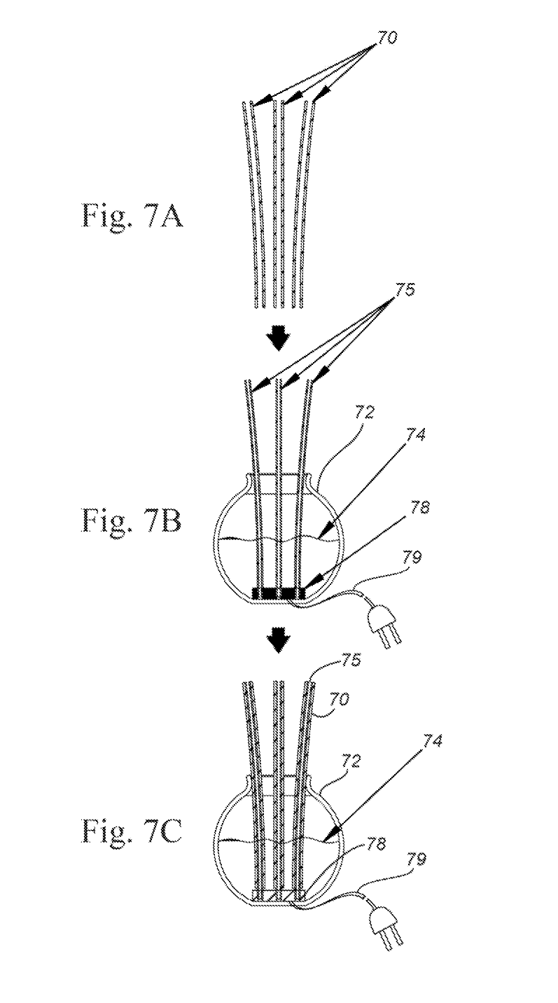

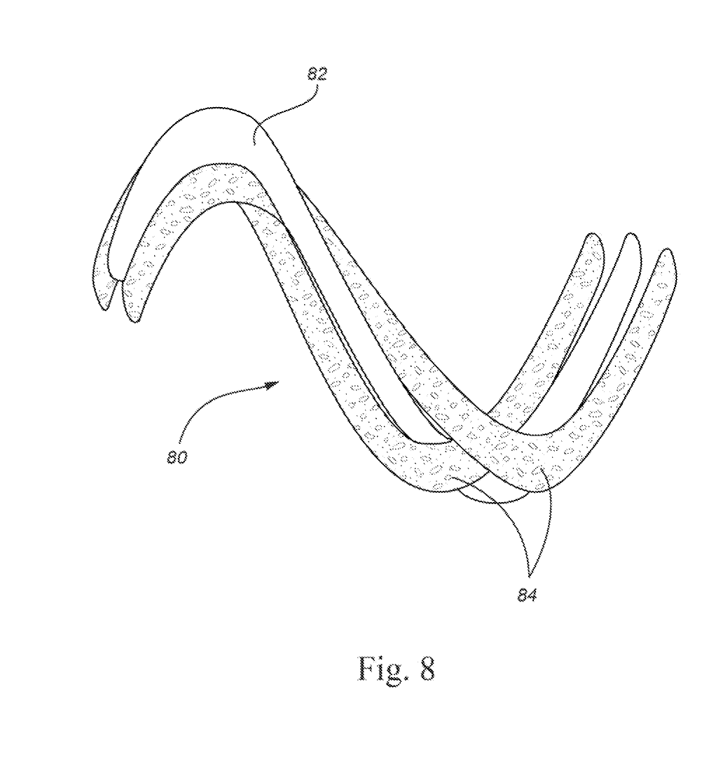

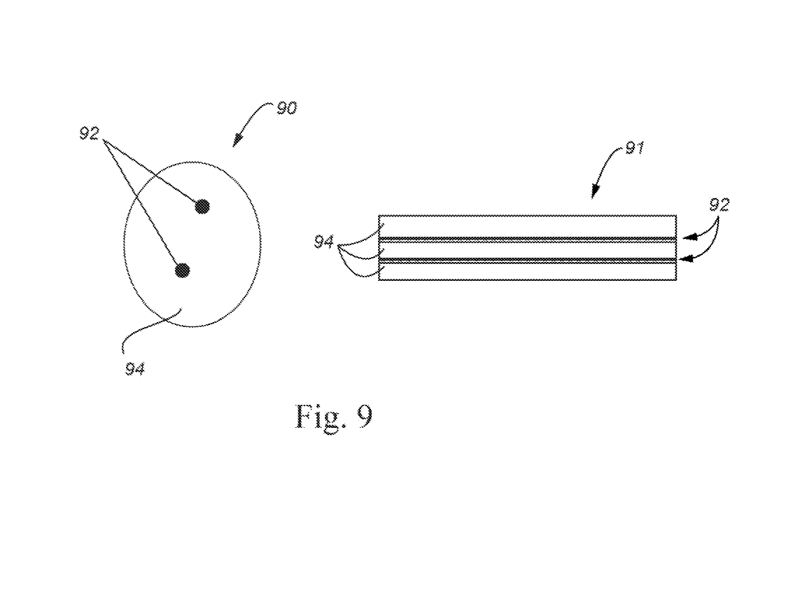

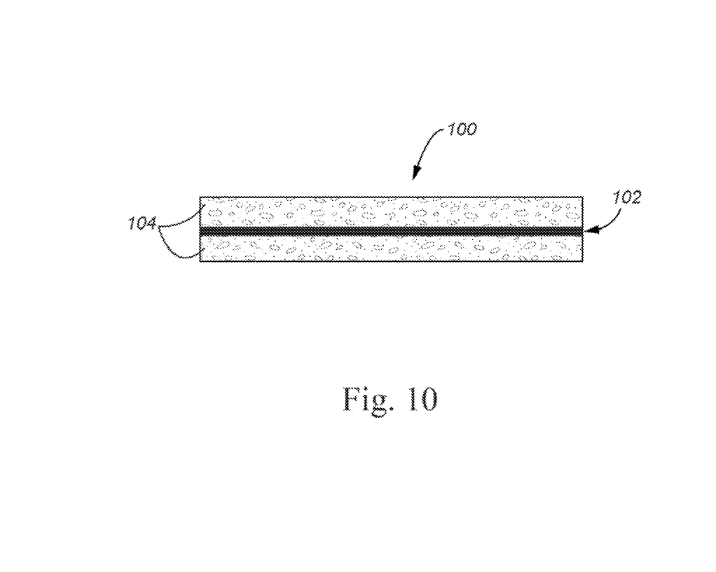

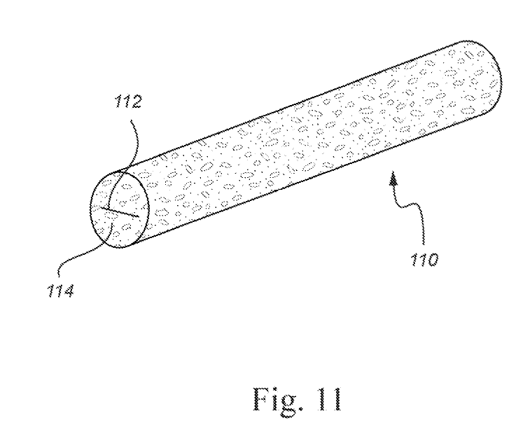

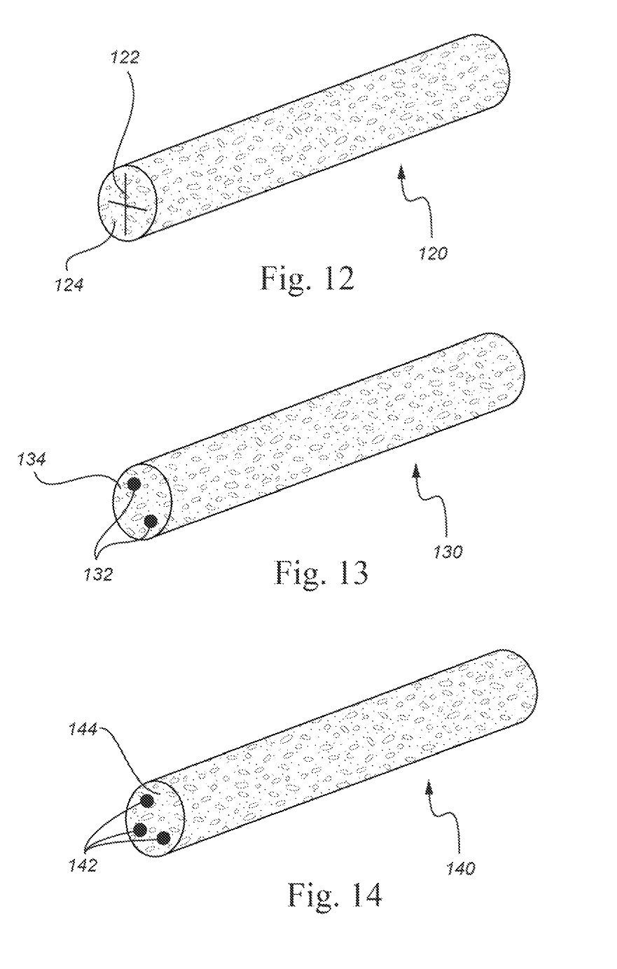

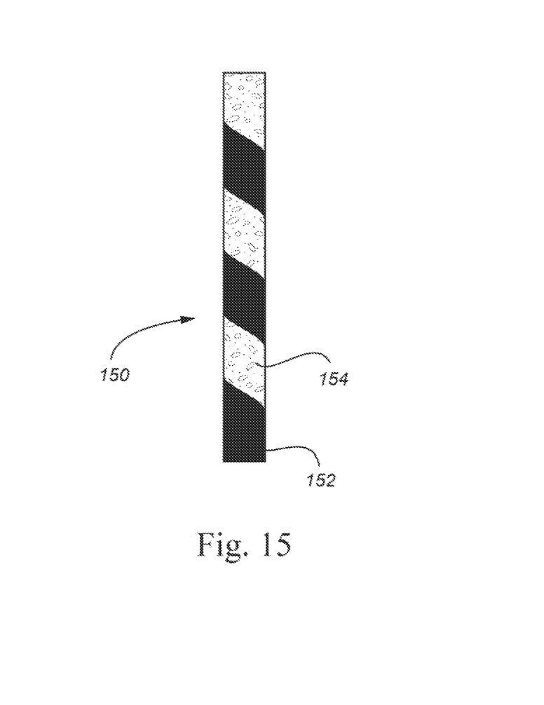

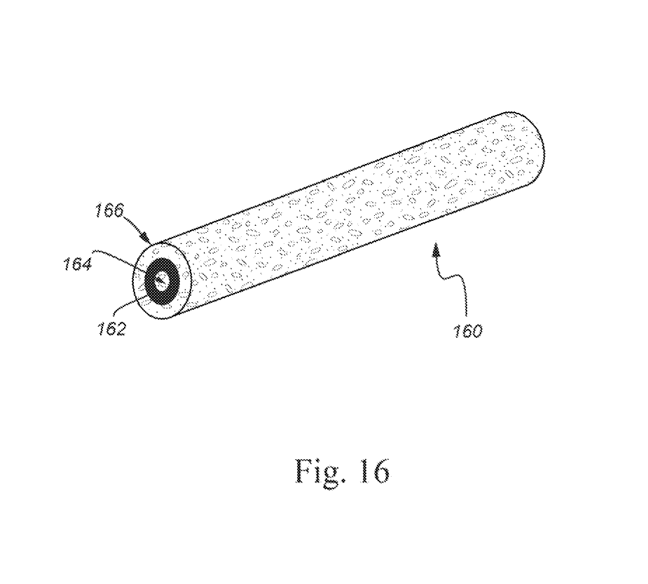

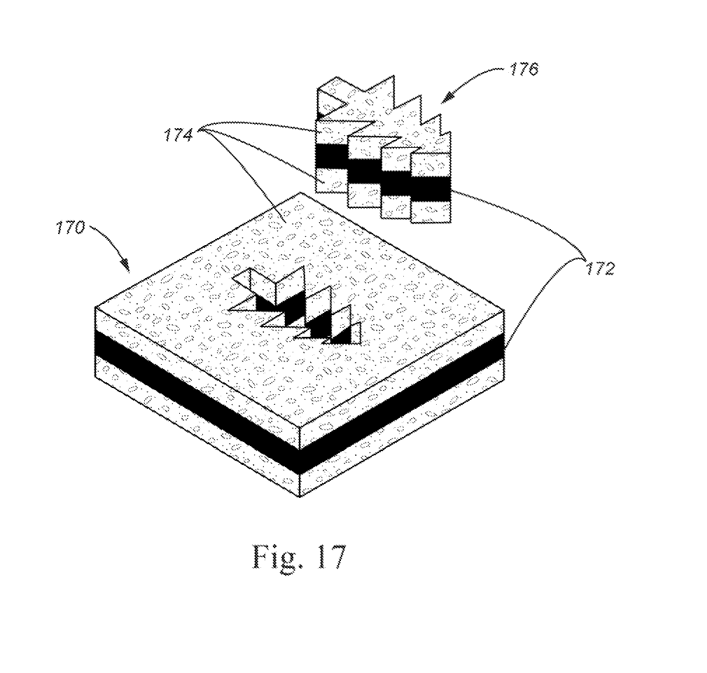

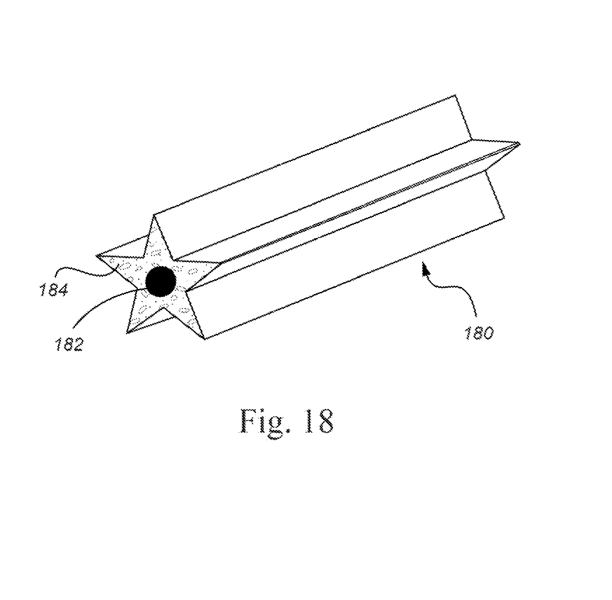

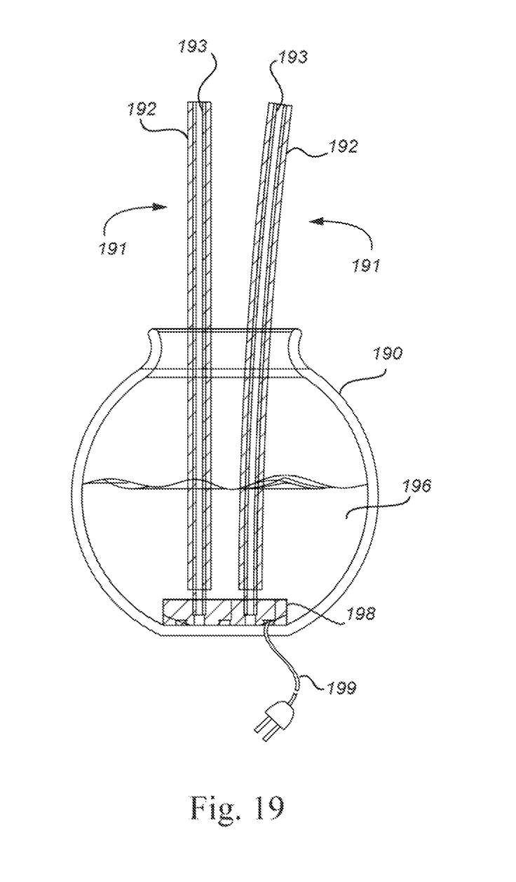

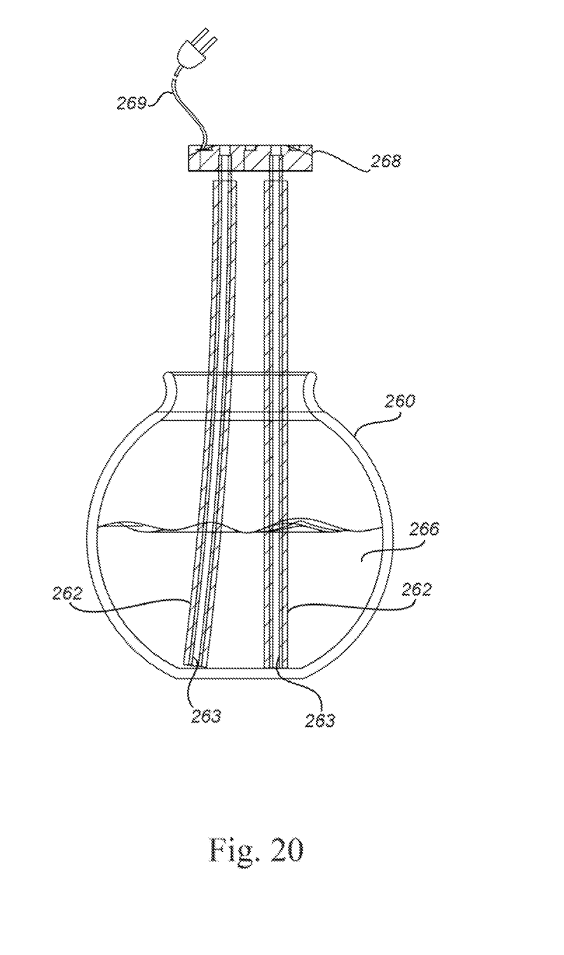

### DETAILED DESCRIPTION

The present invention provides conductive composite wicks, devices containing conductive composite wicks, and methods for wicking and evaporating a liquid in a container of vaporizable materials, such as an air freshener, a perfume, a disinfectant, an insect repellant or an insecticide. These conductive composite wicks are also capable of delivering difficult to vaporize materials including but not limited to organic solvents with low vapor pressures such as dipropylene glycol (DPG).

### Conductive Composite Wicks

These conductive composite wicks comprise a porous wicking element coupled to a conductive element for use in releasing vaporizable materials from devices such as an air freshener device or a device for use in an insect control system. In some embodiments, porous wicking elements may comprise sintered porous plastic. In other embodiments, porous wicking elements may comprise fibrous materials.

Conductive elements may be electrically conductive, thermally conductive or both electrically conductive and thermally conductive. In some embodiments, conductive elements comprise carbon. In other embodiments, conductive elements comprise a metal or metallic alloy. Conductive elements are coupled to a power source for electrical or thermal conductivity.

The conductive composite wicks of the present invention comprise a porous wicking element and a conductive element. In one embodiment, the conductive element on one end of the conductive composite wick is connected to the heating source or power source inside the reservoir and the other end of the conductive composite wick extends out of the container and is exposed to the air. Both ends of the conductive element of the conductive composite wick can be connected to the heating source, power source or electric circuit. A portion of the conductive composite wick is immersed in the reservoir and the other portion of the conductive composite wick extends out of the container and is exposed to the air. The conductive composite wick can be any shape, such as rod, a curved rod, a branched structure or a specific shape such as a flower.

In one embodiment, conductive composite wicks have a porous wicking element and a metallic conductive element. The porous wicking element and metallic conductive element can have many configurations. In another embodiment, the channel for the metallic conductive element is embedded in the porous wicking element. In yet another embodiment, the channel for the metallic conductive element is located at the surface of the porous wicking element. Non-limiting examples of different configurations of the composite wicks are shown in the figures.

In one embodiment, conductive composite wicks have porous a wicking element and a carbon conductive element. In one embodiment, the carbon conductive element is a carbon fiber conductive element. The porous wicking element and carbon fiber element can have many configurations. In another embodiment, the carbon fiber conductive channel is embedded in the porous wicking element. In yet another embodiment, the carbon fiber conductive channel is located at the surface of porous wicking element. Non-limiting examples of different configurations of the composite wicks are shown in the figures.

Porous Wicking Element

Porous wicking elements of the present invention include different types of open cell porous media. These porous wicking media include but are not limited to open cell foams, felts, felts bounded with thermosetting resins, woven fibers, porous media comprising thermosetting resins and inorganic fillers, extruded plastic hollow tubes, and porous media comprising synthetic and natural cellulose materials. In some embodiments, porous wicking elements comprise porous polymeric materials, including but not limited to, sintered porous polymeric materials. In some embodiments, porous wicking elements comprise sintered porous plastic. In other embodiment, the porous liquid wicking element can be a porous fiber material such as a non-woven or woven fiber, or a fiber made by the process described in U.S. Pat. No. 5,607,766 and U.S. Patent Application 20030211799. The fibers can be staple fibers, continuous fibers, bicomponent fibers and mono-component fibers. The porous wicking element may be solid, tubular, or spiral in configuration. The porous wicking element may flexible or relatively rigid. Factors governing materials suitable for the construction of wicks of the present invention include compatibility with the liquid to be transferred by the wick, wicking rates offered by the material, ease of material processing, material cost, etc.

In some embodiments, sintered polymeric materials of the present invention comprise one or a plurality of plastics. Plastics, as used herein, include flexible plastics and rigid plastics. Flexible plastics, in some embodiments, comprise polymers possessing moduli ranging from about 15,000 N/cm² to about 350,000 N/cm² and/or tensile strengths ranging from about 1500 N/cm² to about 7000 N/cm². Rigid plastics, according to some embodiments, comprise polymers possessing moduli ranging from about 70,000 N/cm² to about 350,000 N/cm² and have tensile strengths ranging from about 3000 N/cm² to about 8500 N/cm².

Plastics suitable for use in sintered polymeric materials of the present invention, in some embodiments, comprise polyolefins, polyamides, polyesters, rigid polyurethanes, polyacrylonitriles, polycarbonates, polyvinylchloride, polymethylmethacrylate, polyvinylidene fluoride, polyethersulfones, polystyrenes, polyether imides, polyetheretherketones, polysulfones, polyethersulfone, polyphenylene oxide, or combinations or copolymers thereof.

In some embodiments, a polyolefin comprises polyethylene, polypropylene, and/or copolymers thereof. The polyethylene can be high density polyethylene (HDPE), very high molecular weight polyethylene (VHMWPE) or ultrahigh molecular weight polyethylene (UHMWPE). The average pore size for the porous plastic wick can range from about 10 microns to about 200 microns, about 20 microns to about 150 microns, or about 30 microns to about 100 microns. The porous plastic wick has an average pore volume from about 10% to about 70%, about 20% to about 60%, or about 30% to about 50%. The average pore size and average pore volume are determined by a mercury porosimetry using the ASTM D4404 method.

Polyethylene, in one embodiment, comprises HDPE. High density polyethylene, as used herein, refers to polyethylene having a density ranging from about 0.92 g/cm³ to about 0.97 g/cm³. In some embodiments, high density polyethylene has a degree of crystallinity (% from density) ranging from about 50 to about 90. HDPE has a molecular weight between about 100,000 Daltons (Da) to 500,000 Da.

In another embodiment, polyethylene comprises UHMWPE. UHMWPE, as used herein, refers to polyethylene having a molecular weight greater than 1,000,000, in some embodiments between 3,000,000 Da and 6,000,000 Da.

In another embodiment, polyethylene comprises very high molecular weight polyethylene (VHMWPE). Very high molecular weight polyethylene, as used herein, refers to polyethylene having a molecular weight greater than 300,000 Da and less than 1,000,000 Da.

In some embodiments wherein a wick of the present invention comprises a sintered polymeric material, the wick is produced by providing a plurality of plastic particles in a mold, the mold comprising a cavity having the desired shape of the wick. The plurality of plastic particles are disposed in the mold and sintered to produce a wick of the present invention. Particles of any of the plastics described herein can be sintered into a wick of the present invention.

Plastic particles, in some embodiments, are sintered at a temperature ranging from about 200° F. to about 700° F. In some embodiments, plastic particles are sintered at a temperature ranging from about 300° F. to about 500° F. The sintering temperature, according to embodiments of the present invention, is dependent upon and selected according to the identity of the plastic particles. Appropriate sintering temperatures are known to one of ordinary skill in the art.

Plastic particles, in some embodiments, are sintered for a time period ranging from about 30 seconds to about 30 minutes. In other embodiments, plastic particles are sintered for a time period ranging from about 1 minute to about 15 minutes or from about 5 minutes to about 10 minutes. In some embodiments, the sintering process comprises heating, soaking, and/or cooking cycles. Moreover, in some embodiments, sintering of plastic particles is conducted under ambient pressure (1 atm). In other embodiments, sintering of plastic particles is conducted under pressures greater than ambient pressure.

In another embodiment, a wick comprises a fibrous material. Fibrous materials, according to some embodiments, comprise monocomponent fibers, bicomponent fibers, or combinations thereof. Monocomponent fibers suitable for use in embodiments of the present invention, in some embodiments, comprise polyethylene, polypropylene, polystyrene, nylon-6, nylon-6,6, nylon 12, copolyamides, polyethylene terephthalate (PET), polybutylene terephthalate (TBP), co-PET, or combinations thereof. Monocomponent fibers suitable for use in embodiments of the present invention, in some embodiments, may be biodegradable. Monocomponent fibers suitable for use in embodiments of the present invention, in some embodiments, may be colored, such as colored acrylic fibers.

Synthetic fiber materials that can be used to make the porous wicking elements of the present invention may be biodegradable or non-biodegradable.

Synthetic biodegradable monocomponent fibers include but are not limited to the following: poly lactic acid (PLA), polyhydroxyalkanoates (PHA), polyhydroxybutyrate-valerate (PHBV), and polycaprolactone (PCL).

Bicomponent fibers suitable for use in the porous wicking elements, according to some embodiments of the present invention, comprise polypropylene/polyethylene terephthalate (PET); polyethylene/PET; polypropylene/Nylon-6; Nylon-6/PET; copolyester/PET; copolyester/Nylon-6; copolyester/Nylon-6,6; poly-4-methyl-1-pentene/PET; poly-4-methyl-1-pentene/Nylon-6; poly-4-methyl-1-pentene/Nylon-6,6; PET/polyethylene naphthalate (PEN); Nylon-6,6/poly-1,4-cyclohexanedimethyl (PCT); polypropylene/polybutylene terephthalate (PBT); Nylon-6/co-polyamide; polylactic acid/polystyrene; polyurethane/acetal; polylactic acid (PLA) copolymer/polylactic acid (PLA), and soluble copolyester/polyethylene. Biocomponent fibers, in some embodiments, comprise those disclosed in U.S. Pat. Nos. 4,795,668; 4,830,094; 5,284,704; 5,509,430; 5,607,766; 5,620,641; 5,633,032; and 5,948,529.

Bicomponent fibers, according to some embodiments of the present invention, have a core/sheath or side by side cross-sectional structure. In other embodiments, bicomponent fibers have an islands-in-the-sea, matrix fibril, citrus fibril, or segmented pie cross-sectional structure. Bicomponent fibers comprising core/sheath cross-sectional structure and suitable for use in embodiments of the present invention are provided in Table I.

TABLE 1
Bicomponent Fibers

| Sheath | Core |
|---|---|
| polyethylene (PE) | polypropylene (PP) |
| ethylene-vinyl acetate copolymer | polypropylene (PP) |
| (EVA) | |
| polyethylene (PE) | polyethylene terephthalate (PET) |
| polyethylene (PE) | polybutylene terephthalate (PBT) |
| polypropylene (PP) | polyethylene terephthalate (PET) |
| polypropylene (PP) | polybutylene terephthalate (PBT) |
| polyethylene (PE) | Nylon-6 |
| polyethylene (PE) | Nylon-6,6 |
| polypropylene (PP) | Nylon-6 |
| polypropylene (PP) | Nylon-6,6 |
| Nylon-6 | Nylon-6,6 |
| Nylon-12 | Nylon-6 |
| copolyester (CoPET) | polyethylene terephthalate (PET) |
| copolyester (CoPET) | Nylon-6 |
| copolyester (CoPET) | Nylon-6,6 |
| glycol-modified PET (PETG) | polyethylene terephthalate (PET) |
| polypropylene (PP) | poly-1,4-cyclohexanedimethyl (PCT) |
| polyethylene terephthalate (PET) | poly-1,4-cyclohexanedimethyl (PCT) |
| polyethylene terephthalate (PET) | polyethylene naphthalate (PEN) |
| Nylon-6,6 | poly-1,4-cyclohexanedimethyl (PCT) |
| polylactic acid (PLA) | polystyrene (PS) |
| polyurethane (PU) | acetal |
| polylactic acid (PLA) copolymer | polylactic acid (PLA) |

In some embodiments, fibers comprise continuous fibers. In other embodiments, fibers comprise staple fibers. In one embodiment, for example, a fiber of a fibrous material comprises a staple bicomponent fiber. Staple fibers, according to some embodiments, have any desired length. In some embodiments, fibrous materials are woven or non-woven. In one embodiment, a fibrous material is sintered. In one embodiment, fibrous wicks are optionally colored. In another embodiment, the fibers are optionally dyed before use in formation of a conductive composite wick.

In some embodiments, a porous wicking element has a length up to about 12 inches. In some embodiments, a porous wicking element has a length of at least one inch. In other embodiments, a porous wicking element has a length ranging from about 2 inches to about 12 inches. A porous wicking element, according to some embodiments, has a length less than about 1 inch or greater than about 12 inches. Moreover, the body of a porous wicking element, in some embodiments, has width or diameter of up to about 0.5 inches. In some embodiments, the cross-sectional diameter of a tapered or recessed wick end is at least 0.05 inch.

In some embodiments, the porous wicking element may be biodegradable. The porous wicking element may be hydrophilic. The porous wicking element may be biodegradable and hydrophilic. The term biodegradable is used in this application to indicate that a component of the porous wicking element is biodegradable. In one embodiment, the wt % of the component of the porous wicking element that is biodegradable is at least 40%, at least 50%, at least 60%, at least 70%, at least 80% or at least 90% of the total weight of the porous wicking element. In one embodiment, the major component of the porous wicking element is biodegradable.

The conductive composite wicks of the present invention may be made from different materials and may be used for delivering a vaporizable material into an environment, such as a room environment. Vaporizable materials include aqueous based fragrances in this invention and are fragrance formulations that have a water composition over 50% by weight. The conductive composite wicks of the present invention may also deliver non-aqueous based fragrances into an environment. The conductive composite wicks of the present invention may also deliver other vaporizable materials into an environment.

The wicks of the present invention are optionally treated to increase the surface energy of the porous wicking element and improve the wicking rate. One way to increase this surface energy is to treat porous wicking elements using plasma. This can be a batch process at low pressure or an inline process at or above atmospheric pressure. A number of gases can be energized to react with the surface of the fiber to create hydrophilic moieties and improve hydrophilicity. Gases include, but are not limited to, oxygen, air, nitrogen, argon and a combination thereof. Various exposure times, pressures, and energies are used during the plasma process depending on the desired product requirements. The wicks of the present invention may be optionally activated by employing finishing agents for improved hydrophilicity. In one embodiment, finishing agents may be applied to the bicomponent fibers. In another embodiment, finishing agents are present in the commercially available bicomponent fibers or monocomponent fibers.

Conductive Elements

The conductive elements in the composite wicks may be thermally and/or electrically conductive. In different embodiments, the conductive elements can be metals, metal alloys, or carbon.

In some embodiments, the conductive element is metal. The metal could be selected from aluminum, copper, iron, steel or zinc. In some embodiments, the conductive element is a metal alloy. These alloys comprise one or more of these metal elements, such as steel or stainless steel. Alloys include but are not limited to Kanthal alloys, (FeCrAl), nichrome 80/20 alloys (80% nickel, 20% chromium), and cupronickel alloy (CuNi). Other materials that may be used as conductive elements are molybdenum disilicide (MoSi2), barium titanate and lead titanate.

In one embodiment, the conductive element may be a metal rod, a hollow metal tube, or a metal wire. In one embodiment, the metal element of the composite wick has preset electrical resistance and functions as a resistor. The metal elements generate heat when a current is applied to the metal element of the composite wick. The heat generated by the metal element promotes the wicking of the vaporizable material through the porous wick and release of the vaporizable material into the air.

In one embodiment, a metal rod is embedded in the porous liquid wicking media to form the composite wick. In another embodiment of the composite wick, the metal component is a hollow tube and the porous liquid wicking media is placed over the hollow tube. In yet another embodiment of the composite wick, the metal component is a metal wire or screen and the metal wire or screen is wrapped on the outer surface of porous liquid wicking media.

In some embodiments, a metal rod, tube or wire has a length up to about 12 inches. In some embodiments, a metal rod, tube or wire has a length of at least one inch. In other embodiments, the metal rod, tube or wire has a length ranging from about 2 inches to about 12 inches. The metal rod, tube or wire, according to some embodiments, has a length less than about 1 inch or greater than about 12 inches. Moreover, the body of metal rod, tube or wire, in some embodiments, has a width or a diameter from about 0.01 inch up to about 0.25 inches. In one embodiment the metal component can be longer than the wicking element. In another embodiment, the metal element is shorter than the wicking element. The electric conductivity for the metal component should be greater than 1×10³ (Siemens per meter (S/m)) at 20° C., greater than 1×10⁴ (S/m) at 20° C., or greater than 1×10⁵ (S/m) at 20° C.

In another embodiment, the conductive element is carbon. In another embodiment, the conductive element is carbon fiber. In different embodiments, the carbon fiber can be in tow, yarn, rod, sheet or sleeve form. In another embodiment, carbon fibers can be graphite fibers. Carbon fibers generally have diameter from 0.001 to 0.050 mm. In different embodiments, carbon fibers in this invention have a carbon content of up to 99%, or from 90% to 99%, or from 95% to 99%. Conductive carbon fibers in the conductive composite wicks can be from 0.1% to 90% by weight, from 1% to 50% by weight, from 2% to 30% by weight or from 5% to 20% by weight of the entire wick. The amount of carbon may be varied to change the resistance of the wick. In this manner, wicks with different amounts of carbon may require different amounts of power to release vaporizable material. Carbon fibers are commercially available, for example, from Fibre Glast Development Corp. (Brookville, Ohio); Zoltek Inc. (St. Louis, Mo.); Toho Tenax America, Inc. (Rockwood, Tenn.). Carbon fiber sheet, tube and rod can be purchased from Graphitestore.com Inc. (Buffalo Grove, Ill.).

In some embodiments, the conductive elements in the conductive composite wick can be metal coated fibers, such as nickel-coated carbon fibers. This type of fiber can be purchased from Toho Tenax America, Inc. (Rockwood, Tenn.).

In one embodiment, the conductive carbon element may be a rod, a hollow tube, or a wire. A rod or tube may be embedded in the porous wicking media. The porous wicking media may be placed over the tube or rod, or inserted onto a wire. A wire may also be applied to the inside or outside surfaces of the porous wicking media. Wires may be straight, curved or spiral in configuration. A spiral wire configuration may have an inner diameter to permit insertion of the porous wicking media into the inner diameter of the spiral wire.

In yet another embodiment, the conductive carbon fiber element may be tow or a yarn, and the carbon fiber tow or yarn is embedded in the porous wicking media. In another embodiment, the carbon fiber element can be twisted together with porous wicking media and located on the surface of porous wicking media.

In one embodiment, the conductive carbon fiber element of the conductive composite wick has preset electrical resistance and functions as a resistor. The carbon fiber elements generate heat when a current is applied to the carbon fiber element of the conductive composite wick. The heat generated by the carbon fiber element promotes the wicking of the vaporizable material through the porous wick and release of the vaporizable material into the air.

In another embodiment, the carbon fiber is in a sheet or sleeve form and the carbon fiber is wrapped on the outer surface of porous liquid wicking media.

In one embodiment of a conductive composite wick, carbon fiber tow wraps around other non conductive fiber materials, and fiber material in this case may be loose fibers or sintered fibers, the loose fibers having a structure like a writing instrument reservoir or cigarette filter and the wicks are optionally wrapped with a layer of non-porous skin.

In another embodiment of a conductive composite wick, carbon fibers are wrapped around by other non conductive fiber materials, and fiber material in this case may be loose fibers, the loose fibers having a structure like a writing instrument reservoir or cigarette filter and the wicks are wrapped with a layer of non-porous skin.

In some embodiments, a conductive composite wick has a length up to about 12 inches. In other embodiments, a conductive composite wick has a length of at least one inch. In another embodiment, the conductive composite wick has a length ranging from about 2 inches to about 12 inches. The conductive composite wick, according to some embodiments, has a length less than about 1 inch or greater than about 12 inches.

In some embodiments, the carbon fiber element has a width or a diameter from about 0.01 inch up to about 0.255 inches. In one embodiment the carbon fiber component can be longer than the wicking element. In another embodiment, the carbon fiber component is shorter than the wicking element.

In one embodiment, a composite wick contains multiple carbon fiber conductive channels. The resistance of carbon fibers contained in channels inside or on the outside of the wicking element is from 0.01 ohms to 1000 ohms, from 0.1 to 100 ohms or from 1 to 10 ohms for 3 inch long carbon fibers. Conductivity is the inverse of resistance. Resistance was measured with a multimeter connected to the two ends of the carbon fibers in or on the wick. The conductivity of the carbon fiber channels can be controlled by the diameter of carbon fibers in the carbon fiber channels. The electric conductivity for the carbon fiber materials should be greater than 1×10² (Siemens per meter (S/m)) at 20° C., greater than 1×10³ (S/m) at 20° C., or greater than 1×10⁴ (S/m) at 20° C.

In different embodiments, the bicomponent binding fiber in the conductive composite wick has a diameter from 1 micron to 50 microns. In other embodiments, the carbon fiber has a diameter from 1 micron to 50 microns. In various embodiments, the density of the resulting conductive composite wick can vary from 5 g/meter.cm²) to 50 g/meter.cm². The fiber wicks of the present invention have a pore size in the range of about 10 microns to about 200 microns, about 20 microns to about 150 microns or about 30 microns to about 100 microns. The fiber wicks of the present invention have a pore volume in the range of about 40% to about 95%, about 50% to about 90% or about 60% to about 80%. The pore size and pore volume are determined by a mercury porosimetry using the ASTM D4404 method.

In one embodiment, the conductive composite wick may be colored. In another embodiment, bicomponent fibers in the conductive composite wick are colored. In yet another embodiment, the porous wicking element in the conductive composite wick contains colored monocomponent fiber. In another embodiment, the wicking element comprises black acrylic fibers. Monocomponent fibers may also be dyed before use in formation of the conductive composite wick.

In one embodiment, the bicomponent binding fiber in the conductive composite wick in this invention can be biodegradable, such polylactic acid (PLA)/PLA bicomponent fiber.

### Process of Making Conductive Composite Wick Having Metal Conductive Channels

One component in the bicomponent fiber has a lower melting temperature than another component in the bicomponent fiber. Bicomponent fibers can be fused together by melting the lower melting temperature component thereby forming a porous media with void spaces (pores) between the fibers.

In one embodiment, the conductive composite wicks are made from a bicomponent fiber sliver and a metal wire. The bicomponent fiber sliver and metal wire are drawn together and subsequently subjected to heat and pressure in an oven pultrusion process. A die on the output side of the oven forms rods of desired diameter that are subsequently cut into wicks. The majority of the fibers in the conductive composite wick are composed of the bicomponent synthetic fibers (about 51 wt % to about 95 wt %). The bicomponent synthetic fibers may be at least more than 50 wt %, 60 wt %, 70 wt %, 80 wt %, 90 wt % or 95 wt % of the weight of the wick. The minor component of the fiber in the conductive composite wick is a metal wire.

In another embodiment, the conductive composite wicks are made from a bicomponent fiber sliver and a metal wire. The bicomponent fiber sliver and metal wire are drawn together, with the metal wire located at the center of bicomponent fiber slivers and subsequently subjected to heat and pressure in an oven pultrusion process. A die on the output side of the oven forms rods of desired diameter that are subsequently cut into wicks. The majority of the fibers in the conductive composite wick are composed of the bicomponent synthetic fibers (about 51 wt % to about 95 wt %). The bicomponent synthetic fibers may be at least more than 50 wt %, 60 wt %, 70 wt %, 80 wt %, 90 wt % or 95 wt % of the weight of the wick. The minor component in the conductive composite wick is metal wire. In one embodiment, the channel containing the conductive metal wire is in the center of the conductive composite wick.

In yet another embodiment, the conductive composite wicks are made from a continuous bicomponent fiber yarn and metal wire. The bicomponent fiber yarn and metal wire are drawn together and subsequently subjected to heat and pressure in an oven pultrusion process. A die on the output side of the oven forms rods of desired diameter that are subsequently cut into wicks. The majority of the fibers in the conductive wick are composed of the bicomponent synthetic fibers (about 51 wt % to about 95 wt %). The bicomponent synthetic fibers may be at least more than 50 wt %, 60 wt %, 70 wt %, 80 wt %, 90 wt % or 95 wt % of the weight of the wick. The minor component of the fiber in the conductive wick is a metal wire.

In still another embodiment, the conductive wicks are made from a continuous bicomponent fiber yarn and a continuous metal wire. The bicomponent fiber yarn and metal wire drawn together, with the metal wire located at the center of bicomponent fiber yarn and subsequently subjected to heat and pressure in an oven pultrusion process. A die on the output side of the oven forms rods of desired diameter that are subsequently cut into wicks. The majority of the fibers in the conductive wick are composed of the bicomponent synthetic fibers (about 51 wt % to about 95 wt %). The bicomponent synthetic fibers may be at least more than 50 wt %, 60 wt %, 70 wt %, 80 wt %, 90 wt % or 95 wt % of the weight of the wick. The minor component of the fiber in the conductive composite wick is metal wire. The channel containing the conductive metal wire is at the center of conductive composite wick.

### Process of Making Conductive Composite Wick Having Carbon Fiber Conductive Channels

One component in the bicomponent fiber has a lower melting temperature than another component in the bicomponent fiber. Bicomponent fibers can be fused together by melting the lower melting temperature component thereby forming a porous media with void spaces (pores) between the fibers.

In one embodiment, the conductive composite wicks are made from a bicomponent fiber sliver and a monocomponent carbon fiber tow. The bicomponent fiber sliver and carbon fiber tow are drawn together and subsequently subjected to heat and pressure in an oven pultrusion process. A die on the output side of the oven forms rods of desired diameter that are subsequently cut into wicks. The majority of the fibers in the conductive composite wick is composed of the bicomponent synthetic fiber (about 51 wt % to about 95 wt %). The bicomponent synthetic fiber may be at least more than 50 wt %, 60 wt %, 70 wt %, 80 wt %, 90 wt % or 95 wt % of the weight of the wick. The minor component of the fiber in the conductive composite wick is a monocomponent carbon fiber.

In another embodiment, the conductive composite wicks are made from a bicomponent fiber sliver and a monocomponent carbon fiber tow. The bicomponent fiber sliver and carbon fiber tow are drawn together, with the carbon fiber located at the center of bicomponent fiber slivers and subsequently subjected to heat and pressure in an oven pultrusion process. A die on the output side of the oven forms rods of desired diameter that are subsequently cut into wicks. The majority of the fibers in the conductive composite wick is composed of the bicomponent synthetic fiber (about 51 wt % to about 95 wt %). The bicomponent synthetic fiber may be at least more than 50 wt %, 60 wt %, 70 wt %, 80 wt %, 90 wt % or 95 wt % of the weight of the wick. The minor component of the fiber in the conductive composite wick is a monocomponent carbon fiber. In one embodiment, the channel containing the conductive carbon fiber is in the center of the conductive composite wick.

In yet another embodiment, the conductive composite wicks are made from a continuous bicomponent fiber yarn and a monocomponent carbon fiber tow. The bicomponent fiber yarn and carbon fiber tow are drawn together and subsequently subjected to heat and pressure in an oven pultrusion process. A die on the output side of the oven forms rods of desired diameter that are subsequently cut into wicks. The majority of the fibers in the conductive wick are composed of the bicomponent synthetic fiber (about 51 wt % to about 95 wt %). The bicomponent synthetic fiber may be at least more than 50 wt %, 60 wt %, 70 wt %, 80 wt %, 90 wt % or 95 wt % of the weight of the wick. The minor component of the fiber in the conductive wick is a monocomponent carbon fiber.

In another embodiment, the conductive wicks are made from a continuous bicomponent fiber yarn and a monocomponent carbon fiber tow. The bicomponent fiber yarn and carbon fiber tow are drawn together, with the carbon fiber tow located at the center of bicomponent fiber yarn and subsequently subjected to heat and pressure in an oven pultrusion process. A die on the output side of the oven forms rods of desired diameter that are subsequently cut into wicks. The majority of the fibers in the conductive wick are composed of the bicomponent synthetic fiber (about 51 wt % to about 95 wt %). The bicomponent synthetic fiber may be at least more than 50 wt %, 60 wt %, 70 wt %, 80 wt %, 90 wt % or 95 wt % of the weight of the wick. The minor component of the fiber in the conductive composite wick is a monocomponent carbon fiber. The channel containing the conductive carbon fiber is at the center of conductive composite wick.

In another embodiment the synthetic bicomponent fiber and conductive carbon fiber materials used to make the conductive fiber wicks of the present invention were carded into sliver. The sliver was bonded together by using an oven pultrusion process. The synthetic bicomponent fibers were composed of a concentric sheath and core material. To facilitate sintering, the sheath material was of a lower melting point than the core material. The oven temperature was set between the melting temperature of the sheath and core and the oven thermally bonded (melted) the sheath material of the bicomponent fibers to other bicomponent fibers. This process produced a cylindrical sintered porous matrix. A die compressed and shaped this matrix into rods that were subsequently air cooled and cut to desired length. In this case, the conductive channels containing the carbon fibers are uniformly distributed in the conductive wick.

The fiber materials in the conductive composite wicks are bonded together by using an oven pultrusion process. The oven thermally bonds (melts) the sheath material of the bicomponent fibers to other bicomponent fibers and to the non binding monocomponent carbon fibers that do not melt during the pultrusion process. This process produces a cylindrical sintered porous matrix. A die compresses and shapes this matrix into rods that are subsequently air cooled and cut to length.

The conductive fiber wick can have other shapes such as a sheet or triangle or other profile by changing the die shape in the heating oven, as known to one of ordinary skill in the art in the fiber industry.

The conductive composite wick can also have different wicking properties for the liquids by controlling the surface energy of the porous wicking element of the conductive wick. The processes may include adding surfactant, using sizing agents, or plasma or corona treatment. These processes are known to one of ordinary skill in the art.

### Plasma Treatment

Optionally, the wicking elements in the conductive composite wicks are plasma treated. Plasma treatment could be any one of commonly employed industrial plasma processes, such as radiofrequency (RF) or microwave plasma. The plasma could also be a low pressure or normal pressure air plasma process. In this specific application, plasma is a low pressure, gas plasma treatment process. The wicking elements are placed in a chamber for a specified time, energy level, and gas flow rate. The plasma process makes the wicks more hydrophilic. Gases could be oxygen, nitrogen, argon, hydrogen and any combination thereof. Other molecules, such as alcohol or acrylic acids also could be used in the plasma chamber to make the polymer more hydrophilic. The gas flow rate is controlled to maintain the chamber at a pressure about 100 mtorr and treatment time generally was a few minutes to 30 minutes. It is widely known that plasma treatment conditions depend on the machine design, sample size, power etc. One of ordinary skill in the art can modify conditions for different component parts and on different plasma machines. A plasma treatment device that feeds inline to the pultrusion process and does not require vacuum conditions and operates at positive pressures (above ambient atmospheric pressure) may be used.

The plasma treatment process creates hydrophilic moieties on the surface of the fiber molecules. These moieties increase the surface energy of the fiber wicks making them more hydrophilic. The cross sectional area determines the amount of fluid that can be transported through the wicks for a given wick density. Larger diameter wicks can transport more fluid.

### Finishing Agents

Finishing agents are optionally employed to enhance hydrophilicity of the conductive composite wicks. Finishing agents are well known in the textile industry as aiding agents for the fiber process or provide fiber with desired properties, such as water absorption etc. Finishing agents that may be employed were published in the WO/1993/017172, U.S. Pat. No. 4,098,702, and U.S. Pat. No. 4,403,049. Details of the application of finishing agents, including surfactant, to provide textiles with desired properties may be found in “Handbook of Detergents: Formulation” edited by Michael Showell, pages 279-304, CRC Press, 2005. Application of finishes may generally be accomplished by contacting a fiber tow or yarn with a solution or emulsion comprising at least one finishing agent having desirable lubrication, antistatic, wetting, and/or emulsification properties. Other additives such as antioxidants, biocides, anti-corrosion agents, and pH control agents may also be added into the finishes. A suitable fiber finish may also be sprayed or applied directly onto fibers or yarn. Fibers treated with finishing agents are commercially available.

### Conductive Composite Wicks

The conductive composite wicks can have different diameters and lengths depending on the desired application. Multiple conductive composite wicks may be placed in a jar filled with a liquid containing a vaporizable material such as a fragrance. Capillary forces draw the fragrance solution up the composite wicks, aided by the thermally conductive or electrically conductive element in the composite wick. Fragrance is then released by evaporation of the fluid from the surface of the exposed portion of the composite wicks. One example was a conductive composite wick with a slender rod 0.080 to 0.15 inches in diameter and about 8 inches to about 12 inches long. In one embodiment, conductive composite wicks may be made with diameters of about 0.04 inches to about 1 inch using the pultrusion process. In various embodiments, wick diameters may be about 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, or 0.30 inches. In various embodiments, wick diameters may be greater than about 0.30 inches, greater than about 0.40 inches, greater than about 0.50 inches, greater than about 0.60 inches, or greater than about 1.0 inches. The rods can be cut into wicks of any desired length, for example 0.2 inches or greater. In various embodiments, wicks may be equal to or longer than about 2.0 inches, about 3.0 inches, about 4.0 inches, about 5.0 inches, about 6.0 inches, about 7.0 inches, about 8.0 inches, about 9.0 inches, about 10 inches, about 11 inches, about 12 inches, about 13 inches, about 14 inches, about 15 inches, about 20 inches, about 25 inches, about 30 inches, about 48 inches, about 60 inches, or about 72 inches. In some embodiments, the wicks have a length to diameter ratio greater than 40, greater than 50, greater than 60, greater than 70, greater than 80, greater than 90, greater than 100, greater than 150, greater than 200, greater than 250, or greater than 300. The fiber wicks of the present invention have an average pore volume in the range of about 40% to about 95%, about 50% to about 90% or about 60% to about 80%. The porous plastic wicks of the present invention have an average pore volume from about 10% to about 70%, about 20% to about 60%, or about 30% to about 50%.

### Devices

In one embodiment, the devices containing the conductive composite wicks of the present invention comprise a container with a reservoir of vaporizable material, such as a volatile or vaporizable fluid, to be wicked through the porous wicking media, and a heating element connected to an energy source such that the heating source can receive one or more conductive elements, wherein the container is partially open at the top to permit the porous wicking media to exit the container and access the atmosphere outside the container. In one embodiment, the heating element may be placed within the container or in the housing of the container and is connected to an electrical energy source such as a battery or to an AC or DC source through a wire. The heating source can be also placed outside the containers. In different embodiments, the heating sources can be heating blocks or induction heating coils.

In another embodiment, the conductive element of the conductive composite wick is connected to a receptacle within the container or in the housing of the container and the receptacle is connected to an energy source such as a battery or to an AC or DC source through a wire. In this embodiment, the conductive element generates heat due to its resistance and a separate heating element is not used. It is to be understood that the connection of the power source to the carbon in the conductive composite wick may occur anywhere along the length of the conductive component or at either end of the conductive component of the conductive composite wick.

### Vaporizable Material

In some embodiments, a vaporizable material of a vapor dispensing device of the present invention is a liquid. In other embodiments, a vaporizable material is in a gel, paste, or a solid such as, but not limited to, a wax. Vaporizable materials, in some embodiments of the present invention, comprise fragrances. In another embodiment, vaporizable materials comprise deodorants, perfumes, pheromones, disinfectants, insect repellants, insecticide active agents, pharmaceutical agents. or combinations thereof. In some embodiments, vaporizable materials comprise propylene glycol, water, nicotine, pyruvic acid, or polyethylene glycol.

In some embodiments wherein a vaporizable material is in a gel, the gel is constructed by mixing a vaporizable material with an aqueous based solution and a gel forming agent, such as carrageenan and/or carboxymethylcellulose (CMC). In another embodiment, a vaporizable material is mixed with an alcohol based solution and a gel forming agent in the production of a vaporizable gel material.

Various vaporizable materials may be placed in the container for wicking into the atmosphere. Vaporizable materials containing fragrances may be used to enhance the pleasurable odors in an environment. These wicks can wick and release both aqueous and oil based fragrances. Such fragrances may also mask unpleasant odors in an environment. Vaporizable materials containing insect repellants may be used to repel undesirable invertebrates, such as mosquitoes, no see ums, flies, wasps, yellow jackets and hornets from the environment. Vaporizable materials containing insecticides may kill insects in the environment. Vaporizable materials containing both a fragrance and an insect repellant or insecticide may be employed for the dual function of a pleasurable odor and insect repellency or an insect kill.

Additionally, in some embodiments wherein a vaporizable material is in a solid, the solid is constructed by mixing a vaporizable material such as a fragrance, deodorant, disinfectant, insect repellant, and/or insecticide with a liquid wax and subsequently cooling the mixture to solid form. In one embodiment, the mixture is sprayed prior to cooling to form a powder. Waxes suitable for use in solid vaporizable materials can comprise a natural wax, such as hydroxystearate wax, or a petroleum based wax, such as a paraffin, wherein the wax is optionally impregnated in the porous plastic or fiber wick. In some embodiments, polyethylene oxide (PEO) is used as a substrate for a vaporizable material such as a fragrance, deodorant, disinfectant, insect repellants and/or insecticide.

Vaporizable fragrances, disinfectants, deodorants, insect repellants, and insecticides are well known to one of skill in the art and are available from a variety of commercial sources. Common fragrances comprise citrus oils, fruity floral oils, herbal floral oils, lemon oils, orange oils, or combinations thereof. Disinfectants, in some embodiments, comprise denatonium benzoate, hinokitiol, benzthiazolyl-2-thioalkanoic nitriles, alkyl dimethylbenzyl ammonium chlorides, or trichlosan. Insect repellants, in some embodiments, comprise N,N-diethyl-meta-toluamide, citronella oils, or camphor. Additionally, insecticides, in some embodiments, comprise imiprotrin, cypermethrin, bifentrint, or pyrethrins.

Vaporizable materials, in some embodiments, are disposed in a reservoir of the dispenser. In one embodiment, a vaporizable material comprises a liquid. As described herein, a liquid vaporizable material can be transported from the reservoir through the wick for subsequent vaporization or evaporation. In some embodiments vaporization and evaporation is facilitated or accelerated by a heating element adjacent to the wick. In other embodiments, a vaporizable material is disposed on a surface of the wick or otherwise impregnated into the wick. In such embodiments, the wick can serve as the reservoir for the vaporizable material. In one embodiment, for example, a wick is impregnated and/or coated with a solid, such as a wax containing a vaporizable material. In another embodiment, a wick is impregnated and/or coated with a gel or paste comprising a vaporizable material. In some embodiments wherein the wick is impregnated and/or coated with a solid, gel, or paste containing a vaporizable material, the wick serves as a reservoir for the solid, gel, or paste containing the vaporizable material.

In one embodiment, the conductive element of the conductive composite wick conducts the heat from the heating source from the heating component in the housing, heats the liquid in the reservoir and promotes the wicking and release of the vaporizable liquid into the air.

In another embodiment, the heating source is inside the fragrance reservoir or at the internal surface of the reservoir container. One end of the conductive composite wick is connected to the heating source inside the reservoir and another end of the composite wick extends outside the reservoir and into the air. The composite wick can be any shape, such as a rod. The composite wick could also be a branched structure, such as a flower.

In one embodiment, the conductive element of the conductive composite wick is the heating source when electricity is applied.

In one embodiment, the conductive composite wick described in this invention is heated by induction. The metal components in the composite wicks respond to the induction field. The wick temperature is controlled by the induction field. Since the liquid wicking and evaporation rates depend on the temperature, in some embodiments the liquid delivery rates for the composite wick described in this application are controlled by adjusting the induction field intensity. Some commercially available fragrance delivery devices have a heating element in the housing, however this type of heating element only heats a small section of the wick and could not deliver a wide dynamic range. The composite wick described in this application improves the delivery of fragrance or other vaporizable materials.

In another embodiment, the metal component or carbon component in the conductive composite wick described in this application is part of electrical control circuit. If multiple composite wicks are used in a liquid delivery device, individual conductive composite wicks are selectively turned on and turned off by a switch or a program. The multiple conductive composite wicks in this invention are used to selectively change the liquid delivery rate by controlling the number of wicks turned on or turned off. In one embodiment, turning on a wick means the wick is heated and turning off a wick means the wick is not heated. In another embodiment, turning on a wick means the wick has current and turning off a wick means the wick does not have current.

In one embodiment, a composite conductive wick is functionally linked to an electrical circuit so that the circuit controls whether current flows to the composite conductive wick in an on or off manner. The circuit can control the delivery rate of a vaporizable material, such as a fragrance. In this manner, the timing, duration and the frequency of delivery of the vaporizable material may be controlled. Controlling the delivery rate is useful to decrease olfactory adaptation to the vaporizable material or to deliver the vaporizable material at selected times of the day or night. The circuit can also be used to deliver blends of fragrances from composite conductive wicks contained in the same reservoir or in multiple reservoirs.

In another embodiment using multiple conductive composite wicks, different vaporizable substances may be delivered into the environment by selectively turning on or off conductive composite wicks located in different liquid reservoirs. In this embodiment the device could be a fragrance atomizer device described in U.S. Pat. No. 7,622,073. In this case, conductive composite wicks in different fragrance reservoirs can be selectively turned on or off by a switch or a program. In this manner, more than one fragrance may be delivered to the atmosphere in a selectable manner to create blends of fragrances.

The conductive composite wick described in current invention can also be used with piezoelectric atomizer devices described in U.S. Pat. No. 6,450,419 and U.S. Pat. No. 7,622,073. The composite wicks deliver the liquid to the orifice of the piezoelectric atomizer device and do not dampen the vibration of the piezoelectric device.

In yet another embodiment, a conductive element of a conductive composite wick is part of an electrical circuit. This circuit could provide an electrical signal to trigger other functions in the devices. These activities could be rotation of a fan, turning a light on or off, generating a sound or silencing a sound. These functions provide a desired liquid delivery rate, a desired light feature for decorative purposes or a warning signal for operator attention, such as low liquid level in the reservoir.

In another embodiment, the conductive element of a conductive composite wick has a preset electrical resistance and functions as a resistor. The conductive element can generate heat when an AC or DC power is applied to the conductive element of the composite wick. The heat generated by the conductive element promotes the wicking and release of the vaporizable liquid into the air. In another embodiment, the conductive element of the conductive composite wick is connected to a receptacle within the container or in the housing of the container and the receptacle is connected to an energy source such as a battery or to an AC or DC source through a wire. In this embodiment, the conductive element generates heat due to its resistance and a separate heating element is not used.

In one embodiment, the conductive composite wick may be connected to a circuit configured to measure current or conductivity in the wick or liquid in the reservoir. The circuit can activate other functions for the device if a measurement is below or above a predetermined threshold. These activities could be rotation of a fan, turning a light on or off, generating a sound or silencing a sound. These functions provide a desired liquid delivery rate, a desired light feature for decorative purposes or a warning signal for operator attention, such as low liquid level in the reservoir.

In another embodiment, the conductive composite wick may have or be connected to a circuit configured to detect movement or heat from an insect, an animal or a human. Upon detection of such movement of heat, the circuit can initiate flow of power to the heating element in order to begin volatilization of the volatile fluid through the porous wick. A timing device may be optionally coupled to this circuit such that the power flows for a selected period of time after initiation of volatilization. In this embodiment, power and volatile fluid are conserved.

In another embodiment, the conductive composite wick may have or be connected to a circuit configured with a timer. The circuit can initiate flow of power to the heating element in order to begin volatilization of the volatile fluid through the porous wick at a predetermined time and frequency.

In one embodiment, the power for the devices in this invention is alternating current (AC), for example 110 or 220 volts. In another embodiment, the power for the devices in this invention is direct current (DC), for example from a battery, for example a watch battery, a cell phone battery, an A, AA, AAA, C, or D sized battery, or a car battery. The AC can also be converted to DC through an AC-DC circuit.

The power for the devices in this invention may be generated through solar power, using solar cells or photovoltaic cells known to one of ordinary skill in the solar power field.

The device may be used to deliver vaporizable material to a variety of environments, including but not limited to interior and exterior environments. Interior environments include but are not limited to interior rooms, bathrooms, laundry rooms, closets, near waste receptacles, near litter boxes, tents, boats, planes, and motor vehicles. Exterior environments include but are not limited to patios, decks, campsites, tents, picnic areas, athletic fields and lawns.

In one embodiment, when the device is powered by a DC source, the device is portable and does not rely on connection to an outlet with a wire. Such portable devices may be transported anywhere and powered with the DC source, such as a battery, to release vaporizable material into the atmosphere. In one embodiment, this portable device may be placed on a table in a room or transported to another location and placed on a surface. A portable table top delivery device comprising conductive composite wicks that does not require connection to an outlet is convenient and attractive, permitting greater versatility in placing the device at desired locations. These portable devices are useful in a variety of settings, including but not limited to venues such as campsites, picnic areas, yards, decks, patios, athletic fields and facilities, lavatories, portable toilets and urinals.

One embodiment of this invention is a fragrance delivering device having a container, the container have a heating element, a reservoir of fragrance, and conductive composite wick. One end of composite wick connects to the heating element inside the container and another end of conductive composite wick extends into the air. The device can be powered by AC source or DC battery source. The composite wick could be connected to the heating element in many different ways, such as plugging into the holes of heating element, or screwing onto the heating element. In a specific embodiment, the conductive component of the composite wicks is the part of the heating element. At least a portion of the porous wicking element in the conductive composite wick is immersed in the fragrance and other portions of the porous wicking element are in the air.

The present invention includes a method of using the device to deliver vaporizable material to the air. In one embodiment, the method includes: heating the heating element in the container; the heating element heats the conductive component of the conductive composite wick; the porous wicking element wicks the vaporizable material from the reservoir; and the heated conductive element evaporates the vaporizable material inside the porous wicking element.

The present invention includes a method of using the device to deliver vaporizable material to the air. In another embodiment, the method includes: providing electrical power to the conductive element; the conductive element's resistance heats the conductive element as power is applied; the porous wicking element wicks the vaporizable material from the reservoir; and the heated conductive element evaporates the vaporizable material inside the porous wicking element and releases it into the air.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Conductive Composite Wicks with Biodegradable Bicomponent Fiber Sliver and Conductive Carbon Fiber Tow

The conductive composite wicks were made from pultrusion of synthetic sinterable poly(lactic acid) (PLA) or its copolymer in concentric bicomponent fibers (90%) with a continuous carbon fiber tow (10%) (wt %). In a specific embodiment, both core and sheath materials are PLA and the core PLA has a melting temperature higher than the melting temperature of sheath PLA ((Far Eastern Textile Ltd. Hong Kong or China) Ingeo SLN2450CM, 4 denier)). The carbon fiber tow was from Zoltek Inc., (St. Louis, Mo.). It is preferred that the melting temperature difference is more than 10° C., more than 20° C. or more than 30° C. The melting temperature of the polymer can be controlled by manipulation of crystallization, the copolymerization or the blend as known to one of ordinary skill in the art of polymer chemistry.

The biocomponent fiber in the sliver and carbon fiber tow were bonded together by using an oven pultrusion process. The synthetic biodegradable bicomponent fibers were composed of a concentric sheath and core material. To facilitate sintering, the PLA in the sheath material was of a lower melting point than the PLA in the core material. For this synthetic biodegradable bicomponent fiber, the melting point for the PLA sheath was about 132° C. and melting point for the PLA in the core was about 165° C. The oven temperature was controlled based on the manufacturing conditions. The temperature depended on the pultrusion speed and rod diameter. The goal was to provide a sufficient amount of heat to the sinterable bicomponent fiber such that only the sheath of the bicomponent fiber melted but not the core. The bicomponent fiber silver and carbon fiber tow were pultruded through an oven at the temperature of 204-221° C. and compressed through a die at the temperature of 49-66° C. The pultrusion speed was 2.0 to 4.0 inches/seconds. This process produced a cylindrical conductive porous matrix. A die compressed and shaped this matrix into rods that were subsequently air cooled and cut to length.

### Example 2

### Conductive Composite Wicks with Bicomponent Fiber and Carbon Fiber

The conductive composite wicks were made by combining polyethylene/polyester (PE/PET) concentric bicomponent fiber sliver (90%) with carbon fiber tow (10%) (Zoltek Inc, (St. Louis, Mo.). The biocomponent fiber in the sliver and carbon fiber tow were bonded together using an oven pultrusion process. The bicomponent fibers were composed of a concentric sheath and core material. To facilitate sintering, the sheath material was of a lower melting point than the core material. The oven thermally bonded (melted) the sheath material of the bicomponent fibers to other bicomponent fibers and to the non binding fibers. These non binding fibers include monocomponent fibers such as naturally colored cotton. The non-binding fibers generally did not melt and bind to each other. The silver was pultruded through an oven at the temperature of 175-220° C. and compressed through a die at the temperature of 49-66° C. The pultrusion speed was 2.0 to 4.0 inches/seconds. This process produced a cylindrical conductive porous matrix. A die compressed and shaped this matrix into rods that were subsequently air cooled and cut to length.

### Example 3

### Conductive Composite Wicks with Bicomponent Fiber, Conductive Carbon Fiber and Colored Monocomponent Fiber

The conductive composite wicks were made by combining polyethylene/polyester (PE/PET) concentric bicomponent fiber sliver (63%), carbon fiber tow (5%) (Zoltek Inc, (St. Louis, Mo.) and black colored acrylic fiber (32%). The biocomponent fiber in the sliver carbon fiber tow and black colored acrylic monocomponent fiber were bonded together by using an oven pultrusion process. The bicomponent fibers were composed of a concentric sheath and core material. To facilitate sintering, the sheath material was of a lower melting point than the core material. The oven thermally bonded (melted) the sheath material of the bicomponent fibers to other bicomponent fibers, carbon fiber tow and monocomponent black acrylic fiber together. The carbon fibers and monocomponent acrylic fibers generally did not melt and bind to each other in this process. The bicomponent fiber silver, carbon fiber tow and monocomponent black acrylic fiber were pultruded through an oven at the temperature of 175-220° C. and compressed through a die at the temperature of 49-66° C. The pultrusion speed was 2.0 to 4.0 inches/seconds. This process produced a cylindrical conductive porous matrix. A die compressed and shaped this matrix into rods that were subsequently air cooled and cut to length. The wicks made in this process were black in color.

### Example 4

### Fragrance Delivery Devices with Conductive Carbon Fiber Wicks

The conductive composite wicks made as in example 2 were inserted into a container containing a fragrance. The two ends of the conductive wick were connected to wires. The wires were connected to power box with battery. The temperatures of conductive wick were recorded with an IR thermometer (RadioShack) before and after the power was turned on. The wick was at 78° F. before being connected to one AA battery and the temperature was stable. After the wick was connected to one AA battery, the wick temperature increased and reached 106° F. within 3 minutes and stabilized. When the wick was connected to two AA batteries (in series), the wick temperature increased and reached 147.5° F. within 3 minutes and stabilized. The fragrance delivery rate to the environment was related to the wick temperature. As the wick temperature increased, the environment was filled with a stronger odor of the fragrance as reported by individuals in the room.

### Example 5

### Conductive Composite Wick Liquid Evaporation

In this example, the conductive composite wick comprised bicomponent fiber, conductive carbon fiber, and colored monocomponent fiber. The carbon fiber conductive element was embedded in the center of conductive composite fiber wick. The wicks were made by combining polyethylene/polyester (PE/PET) concentric bicomponent fiber sliver (63%) (FiberVisions, Duluth, Ga.), carbon fiber tow (5%) (Zoltek Inc, (St. Louis, Mo.)) and black colored acrylic fiber (32%). The percentages are the weight % of each component. The biocomponent fiber in the sliver carbon fiber tow and black colored acrylic monocomponent fiber were bonded together using an oven pultrusion process. The bicomponent fibers were composed of a concentric sheath and core material. To facilitate sintering, the sheath material was of a lower melting point than the core material. The oven thermally bonded together (melted) the sheath material of the bicomponent fibers to other bicomponent fibers, carbon fiber tow and monocomponent black acrylic fiber. The carbon fibers and monocomponent acrylic fibers generally did not melt and bind to each other in this process. The bicomponent fiber silver, carbon fiber tow and monocomponent black acrylic fiber were pultruded through an oven at a temperature of 175-220° C. and compressed through a die at a temperature of 49-66° C. The pultrusion speed was 2.0 to 4.0 inches/seconds. This process produced a cylindrical conductive porous matrix. A die compressed and shaped this matrix into rods that were subsequently air cooled and cut to length. The wicks made in this process had a black color. The wicks were 0.25 inch in diameter and the carbon fiber conductive channels were located in the center of composite wicks. The electrical resistance for the carbon core in the conductive composite wick was about 12 ohms per foot.

The conductive composite wick with a length of 12 inches was folded in a U shape and placed in a 50 ml Corning conical-bottom disposable plastic tube (Corning, N.Y.). The tube was filled with a vaporizable liquid at around the 40 ml indicator line. The total weight of the tube, liquid and wick was recorded. The wick was connected to a DC power supply (EXTECH Digital single output DC power supply (purchased from Grainger) and an electric potential was applied to the two ends of the wick. The total weight was recorded at different times. The weight difference between the original weight and recorded weight was the amount of liquid vaporized through the system. Table 2 and table 3 present the weight loss data for deionized water with 1% Tween 20®, and dipropylene glycol (DPG). The data were collected with no applied voltage and also with 1.5V, 4.5V and 9V DC power conditions.

TABLE 2
Deionized water with 1% Tween 20 ® weight loss over time under
different electrical power conditions for conductive composite wicks

| No electrical | | 1.5 V, 0.1 amp | | |
|---|---|---|---|---|
| energy applied | | (A) | 4.5 V, 0.28 A | 9 V, 0.58 A |
| | Weight Loss | Weight Loss | Weight Loss | Weight Loss |
| Time (hrs) | (gm) | (gm) | (gm) | (gm) |
| 0 | 0 | 0 | 0 | 0 |
| 1 | 0.3 | 0.4 | 1.3 | 1 |
| 2 | 0.4 | 0.9 | 1.6 | 2 |
| 3 | 0.6 | 1.2 | 2.4 | 3 |
| 4 | 1 | 1.5 | 3.3 | 4 |
| 5 | 1.3 | — | 4.4 | 5 |
| 20 | — | 5.7 | — | 20 |
| 26 | 5.7 | — | 18.6 | 26 |

The results show that the conductive composite wick delivered more water through the wick when a higher electric energy was applied to the conductive element in the conductive composite wick.

TABLE 3
Dipropylene glycol (DPG) weight loss over time under different
electrical power conditions for conductive composite wick.

| No electrical | | | |
|---|---|---|---|
| energy applied | | 4.5 V, 0.28 A | 9 V, 0.58 A |
| Time (hrs) | Weight Loss (gm) | Weight Loss (gm) | Weight Loss (gm) |
| 0 | 0 | 0 | 0 |
| 1 | 0 | 0 | 0.1 |
| 2 | 0 | 0 | 0.3 |
| 3 | 0 | 0 | 0.5 |
| 18 | — | 0 | — |
| 24 | −0.4 | 0 | 3 |
| 69 | −0.8 | — | 8.6 |

The results demonstrate that the conductive composite wick delivered low vapor pressure, hydroscopic DPG at 9V electrical energy. DPG could not be vaporized through traditional wicking methods.

### Example 6

### Conductive Composite Wicks Comprising Porex e-Reed and Conductive Carbon Fiber Wrapped on the Surface of the e-Reed

Porex e-Reeds™ X21193 (Porex, Fairburn, Ga.) were made by combining sinterable polyethylene/polyester (PE/PET) concentric bicomponent fibers (FiberVisions, Duluth, Ga.) with non-sinterable, natural brown cotton fibers (Vreseis Ltd. Trade name: Fox fiber). These materials were blended in a 9:1 ratio and carded into sliver. The lower content brown cotton provided the natural color of the wicks.

The sliver was bonded together using an oven pultrusion process. The bicomponent fibers were composed of a concentric sheath and core material. To facilitate sintering, the sheath material had a lower melting point than the core material. The oven thermally bonded (melted) the sheath material of the bicomponent fibers to other bicomponent fibers and to the non-binding fibers. These non-binding fibers include monocomponent fibers such as naturally colored cotton. The non-binding fibers generally do not melt and bind to each other. The silver was pultruded through an oven at a temperature of 175-220° C. and compressed through a die at a temperature of 49-66° C. The pultrusion speed was 2.0 to 4.0 inches/seconds. This process produced a cylindrical sintered porous matrix. A die compressed and shaped this matrix into rods that were subsequently air cooled and cut 11 inches in length and with a diameter of 0.125 inches.

Two groups of e-Reeds were wrapped with two feet of carbon fiber tow (Zoltek Inc, (St. Louis, Mo.). The electrical resistance for the carbon fiber wrapped around the e-Reed wick was about 10 ohms per foot. Each group had two e-Reeds wrapped together and two groups of e-Reeds were linked together by the carbon fiber tow. Each of the two groups of e-Reeds had two open ends that were connected to an external power source and two closed ends linked by the continuous carbon fiber tow. Two groups of wrapped e-Reeds were placed in a 50 ml Corning Conical-bottom disposable plastic tube. The open ends were above the tube and closed ends were immersed in the liquid. The tube was filled with a vaporizable liquid at about the 40 ml mark line. The total weight of the tube, liquid and wick was recorded. The wick was connected to a DC power supply (EXTECH Digital single output DC power supply purchased from Grainger) and an electric potential was applied to the two open ends of the e-Reed. The total weight was recorded at different times. The weight difference between the original weight and recorded weight was the amount of liquid vaporized through the system. Tables 4, 5 and 6 show the weight loss data for deionized water with 1% Tween 20®, dipropylene glycol methyl ether acetate (DPMA) and dipropylene glycol (DPG), respectively. The data were collected with no voltage applied and under 4.5V and 9V DC power conditions.

TABLE 4
Weight loss of deionized water with 1% Tween 20 ® for carbon fiber
wrapped e-Reed X21193 with and without applied voltage. The data were
collected with no electrical energy applied and under 4.5 V and 9 V DC
power conditions.

| No electrical | | | |
|---|---|---|---|
| energy applied | | 4.5 V, 0.4 A | 9 V, 0.8 A |
| Time (hrs) | Weight Loss (gm) | Weight Loss (gm) | Weight Loss (gm) |
| 0 | 0 | 0 | 0 |
| 1 | 0.4 | 2.2 | 4.6 |
| 2 | 1.3 | 3.5 | 8.3 |
| 3 | 1.9 | 4.9 | 9.9 |
| 4 | — | 6.5 | 13.4 |
| 5 | — | 7.8 | 16 |
| 6 | — | 9.8 | 18.7 |
| 7 | — | 10.8 | — |
| 16 | 5.7 | — | — |
| 23 | 6.4 | 29 | 53.7 |

TABLE 5
Weight loss of dipropylene glycol methyl ether acetate (DPMA) for
carbon fiber wrapped e-Reed X 21193 with and without applied voltage.
The data were collected with no electrical energy applied and under 4.5 V
and 9 V DC power conditions.

| No electrical | | | |
|---|---|---|---|
| energy applied | | 4.5 V, 0.4 A | 9 V, 0.8 A |
| Time (hrs) | Weight Loss (gm) | Weight Loss (gm) | Weight Loss (gm) |
| 0 | 0 | 0 | 0 |
| 1 | 0.1 | 0.2 | 4.1 |
| 2 | 0.2 | 0.4 | 6.7 |
| 3 | 0.4 | 0.6 | 8.0 |
| 4 | 0.4 | 0.8 | 10 |
| 5 | 0.6 | 1.1 | 12.6 |
| 6 | 0.6 | 1.4 | 15.2 |
| 7 | — | 1.6 | — |
| 22 | 1.8 | — | 45.8 |
| 24 | 1.9 | 4.1 | — |

TABLE 6
Weight loss of dipropylene glycol (DPG) for carbon fiber wrapped e-Reed
X 21193 with and without electric potential. The data were collected with
no electrical energy applied and under 4.5 V and 9 V DC
power conditions.

| No electrical energy applied | | 4.5 V, 0.28 A | 9 V, 0.58 A |
|---|---|---|---|
| Time (hrs) | Weight Loss (gm) | Weight Loss (gm) | Weight Loss (gm) |
| 0 | 0 | 0 | 0 |
| 1 | 0 | 0.3 | 0.9 |
| 2 | 0 | 0.6 | 1.4 |
| 3 | −0.2 | 0.8 | 1.6 |
| 4 | 0 | 0.9 | 2.2 |
| 5 | 0 | 1 | 4.2 |
| 6 | 0 | 1.1 | 6.1 |
| 7 | 0 | 1.2 | — |
| 22 | 0 | 1.3 | 9.2 |
| 24 | 0.1 | 1.6 | 9.5 |

The data indicate that conductive composite wicks with e-Reeds and carbon fibers showed electric energy dependent liquid vaporization capability for water, DPMA and DPG. A greater amount of electrical energy evaporated more liquid into the environment.

### Example 7

### Conductive Composite Wicks Comprising a Sintered Porous Plastic Rod and Conductive Carbon Fiber

A Porex sintered porous plastic rod wick (X-5531, Porex, Fairburn, Ga.) 1 foot in length and 0.25 inches in diameter was made by sintering UHMWPE in a mold. The wick had an average pore size of 40 microns and average pore volume of 40%. Two X-5531 wicks were wrapped with two feet of carbon fiber tow (Zoltek Inc, (St. Louis, Mo.). The electrical resistance for the carbon fiber wrapped around the wick was about 10 ohms per foot. Two X-5531 rods were linked together by the carbon fiber tow. The rods had two open ends that were connected to an external power source and the two closed ends were linked by the continuous carbon fiber tow. The two wicks were placed in a 50 ml Corning Conical-bottom disposable plastic tube. The open ends were above the tube and the closed ends were immersed in the liquid. The tube was filled with a vaporizable liquid at about the 40 ml mark line. The total weight of the tube, liquid and sintered porous rod wick were recorded. The wick was connected to a DC power supply (EXTECH Digital single output DC power supply) and an electric potential was applied to the two open ends of the sintered porous plastic wick. The total weight was recorded at different times. The weight difference between the original weight and recorded weight was the amount of liquid vaporized through the system. Table 7, 8 and 9 present the weight loss data for deionized water with 1% Tween 20®, dipropylene glycol methyl ether acetate (DPMA), and dipropylene glycol (DPG), respectively. The data were collected with no applied voltage and under 4.5V and 9V DC power conditions.

TABLE 7
Weight loss data of deionized water with 1% Tween 20 ® for a porous
plastic rod wick wrapped with carbon fiber. The data were collected with
no electrical energy applied and under 4.5 V and 9 V DC
power conditions.

| No electrical | | | |
|---|---|---|---|
| energy applied | | 4.5 V | 9 V |
| Time (hrs) | Weight Loss (gm) | Weight Loss (gm) | Weight Loss (gm) |
| 0 | 0 | 0 | 0 |
| 1 | 0.1 | 2 | 3.4 |
| 2 | 0.1 | 2.7 | 4.8 |
| 3 | 0.2 | 3.9 | 7.4 |
| 4 | 0.5 | 5.5 | 10.5 |
| 5 | 0.7 | 6.8 | 12.8 |
| 6 | 0.9 | 7.6 | 14.6 |
| 7 | 1.6 | 9.1 | 16.7 |

TABLE 8
Weight loss data of dipropylene glycol methyl ether acetate (DPMA) for
porous plastic rod wick wrapped with carbon fiber. The data were
collected with no electrical energy applied and under 4.5 V and
9 V DC power conditions.

| No electrical | | | |
|---|---|---|---|
| energy applied | | 4.5 V | 9 V |
| Time (hrs) | Weight Loss (gm) | Weight Loss (gm) | Weight Loss (gm) |
| 0 | 0 | 0 | 0 |
| 1 | 0.1 | 0.2 | 2 |
| 2 | 0.1 | 0.3 | 2.6 |
| 3 | 0.2 | 0.4 | 4.1 |
| 4 | 0.4 | 0.6 | 5.4 |
| 5 | 0.4 | 0.7 | 6.4 |
| 6 | 0.5 | 0.9 | 7.6 |
| 7 | 0.6 | 1.1 | 8.7 |

TABLE 9
Weight loss data of dipropylene glycol (DPG) for porous plastic rod wick
wrapped with carbon fiber. The data were collected with no electrical
energy applied and under 4.5 V and 9 V DC power conditions.

| No electrical | | | |
|---|---|---|---|
| energy applied | | 4.5 V | 9 V |
| Time (hrs) | Weight Loss (gm) | Weight Loss (gm) | Weight Loss (gm) |
| 0 | 0 | 0 | 0 |
| 1 | 0 | 0 | 0.2 |
| 2 | 0 | 0 | 0.4 |
| 3 | 0 | 0 | 0.6 |
| 4 | −0.1 | 0 | 0.9 |
| 5 | −0.1 | 0 | 1.0 |
| 6 | −0.1 | 0 | 1.2 |
| 7 | −0.1 | 0 | 1.3 |

The data indicate that conductive composite wicks with sintered porous plastic wicks and carbon fibers showed electrical energy dependent liquid vaporization capability for water, DPMA and DPG. Higher electrical energy evaporated more liquid into the environment.

### Example 8

### Conductive Composite Wicks Comprising a Sintered Porous Plastic Rod and Conductive Copper Wire

A Porex sintered porous plastic rod wick (X-5531, Porex, Fairburn, Ga.) 1 foot in length and 0.25 inches in diameter was made by sintering UHMWPE in a mold. The wick had an average pore size of 40 microns and average pore volume of 40%. Two X-5531 rods were wrapped with two feet of copper wire. The copper wire was 1 mm in diameter and had no measurable resistance. One end of each X-5531 rod had an extra 8 inches length of copper wire extending from the end of the rod. Two rods were placed in a 50 ml Corning Conical-bottom disposable plastic tube. The end of the rod with extra copper wire was above the tube and the other end was immersed in the liquid. The tube was filled with a vaporizable liquid at about the 40 ml mark line. The total weight of the tube, liquid and sintered porous rod wick was recorded. The copper wire at the end above the tube was connected to a heated metal plate. The total weight was recorded at different times. The weight difference between the original weight and recorded weight was the amount of liquid vaporized through the system. Table 10 presents the weight loss data for deionized water with 1% Tween 20®. The data were collected under conditions of no externally applied heat and also with the plate set at 105° C. The data indicate that conductive composite wicks with sintered porous plastic wick and copper wire vaporized more water into the environment by heating one end of the conductive copper wire.

TABLE 10
Weight loss data of deionized water with 1% Tween 20 ® for a
porous plastic rod wick wrapped with copper wire. The data were
collected under conditions of no externally applied heating and also with
one end of the copper wire connected to a hot plate set at 105° C.

| | No heat applied | Copper end heated to 105° C. |
|---|---|---|
| Time (hrs) | Weight Loss (gm) | Weight Loss (gm) |
| 0 | 0 | 0 |
| 1 | 0.1 | 0.1 |
| 2 | 0.1 | 0.4 |
| 3 | 0.2 | 0.8 |
| 4 | 0.5 | 2.1 |
| 5 | 0.7 | 2.5 |
| 6 | 0.9 | 3.2 |
| 7 | 1.6 | 4.1 |
| 8 | — | 5.1 |

### Example 9

### Conductive Composite Wick for Aqueous Based Fragrance Delivery

Four e-Reeds (e-Reeds X21193, Porex, Fairburn, Ga.) and two porous plastic rods (X 5531 UHMWPE, Porex, Fairburn, Ga.) were employed, with and without carbon fiber tow. The e-Reeds with carbon fiber conductive elements were the same as disclosed in example 6. The porous plastic rods with carbon fiber conductive elements were the same as disclosed in example 7. The rods were placed in a 50 ml Corning Conical-bottom disposable plastic tube containing liquid. One end of the e-Reeds and one end of the porous plastic rod was immersed in the liquid and the other end of each rod was outside the tube. The wicks were placed into the tube as disclosed in examples 6 and 7. The tube was filled with Honey Vanilla fragrance (Mane, New York, N.Y.) at about the 25 ml mark line. The total weight of the tube, liquid and sintered porous rod wick were recorded. The wick was connected to a DC power supply (EXTECH Digital single output DC power supply purchased from Grainger) and 4.5 V electric potential was applied to the two open ends of the sintered porous plastic wick. No voltage was applied to the e-Reed and porous plastic rod without carbon fiber tow. A tube with fragrance alone was used as control. The total weight was recorded at different times. The weight difference between the original weight and recorded weight was the amount of liquid vaporized through the system. Table 11 lists the weight loss data for the control, four e-Reeds (Porex X 21193), two porous plastic rod (Porex X 5531), composite wick comprising of four e-Reeds (Porex X 21193) with carbon fiber and composite wick comprising two porous plastic rods (Porex X 5531) with carbon fiber.

TABLE 11
Weight loss for the control, four e-Reeds (Porex X 21193), two porous
plastic rods (Porex X 5531), composite wick comprising of four e-Reeds
(Porex X 21193) with carbon fiber and composite wick comprising two
porous plastic rods (Porex X 5531) with carbon fiber over time.

| | | | | | Two porous |
|---|---|---|---|---|---|
| | | | | Four e-Reeds | plastic rods |
| | Control | | | with carbon | with carbon |
| Time | Fragrance | Four | Two porous | fiber at | fiber at |
| (hr) | only | e-Reeds | plastic rods | 4.5 V | 4.5 V |
| 0 | 0 | 0 | 0 | 0 | 0 |
| 1 | 0.0 | 0.3 | 0 | 1 | 0.9 |
| 2 | 0.0 | 0.9 | 0.5 | 1.9 | 2 |
| 3 | 0.0 | 1.2 | 1 | 2.5 | 2.6 |
| 4 | 0.0 | 1.8 | 1.6 | 3.4 | 3.7 |
| 5 | 0.0 | 1.9 | 1.9 | 3.9 | 4.3 |
| 6 | 0.0 | 2.2 | 2.3 | 4.4 | 5 |
| 7 | 0.0 | 2.6 | 2.7 | 5.1 | 5.7 |
| 23.5 | 0.3 | 5.7 | 6.6 | 10.9 | 9.9 |

The data indicate that both conductive composite wicks with e-Reeds and carbon fibers, and conductive composite wicks with sintered porous plastic wick and carbon fibers showed electrical energy dependent, aqueous based, fragrance vaporization capability. Higher amounts of electrical energy evaporated more fragrance into the environment.

### Example 10

### Conductive Composite Wick for Oil Based Fragrance Delivery

Four e-Reeds (e-Reeds X21193, Porex, Fairburn, Ga.) and two porous plastic rods (X 5531 UHMWPE, Porex, Fairburn, Ga.) were employed, with and without carbon fiber tow. The e-Reeds with carbon fiber conductive elements were the same as disclosed in example 6. The porous plastic rods with carbon fiber conductive elements were the same as disclosed in example 7. The rods were placed in a 50 ml Corning Conical-bottom disposable plastic tube containing liquid. One end of the e-Reeds and one end of the porous plastic rod was immersed in the liquid and the other end of each rod was outside the tube. The wicks were placed into the tube as disclosed in examples 6 and 7. The tube was filled with Glade® sweet pea & lilac scented oil at about the 35 ml mark line (SC Johnson, Racine, Wis.). The total weight of the tube, liquid and sintered porous rod wick was recorded. The wick was connected to a DC power supply (EXTECH Digital single output DC power supply purchased from Grainger) and 4.5 V electrical potential was applied to the two open ends of the sintered porous plastic wick. No voltage was applied to the e-Reed and porous plastic rod that did not have carbon fiber tow. A tube with fragrance alone was used as control. The total weight was recorded at different times. The weight difference between the original weight and recorded weight was the amount of liquid vaporized through the system. Table 12 presents the weight loss data for the control, four e-Reeds (Porex X 21193), two porous plastic rods (Porex X 5531), composite wick comprising of four e-Reeds (Porex X 21193) with carbon fiber, and composite wick comprising two porous plastic rods (Porex X 5531) with carbon fiber.

TABLE 12
Weight loss for the control, four e-Reeds (Porex X 21193), two porous
plastic rods (Porex X 5531), composite wick comprising of four e-Reeds
(Porex X 21193) with carbon fiber and composite wick comprising
two porous plastic rods (Porex X 5531) and carbon fiber over time.

| | | | | | Two porous |
|---|---|---|---|---|---|
| | | | | Four e-Reeds | plastic rods |
| | Control | | | with carbon | with carbon |
| Time | Fragrance | Four | Two porous | fiber | fiber |
| (hrs) | only | e-Reeds | plastic rods | 4.5 V | 4.5 V |
| 0 | 0 | 0 | 0 | 0 | 0 |
| 1 | 0.0 | 0.1 | 0.1 | 0.3 | 0.2 |
| 2 | 0.0 | 0.2 | 0.2 | 0.4 | 0.4 |
| 3 | 0.0 | 0.3 | 0.3 | 0.6 | 0.5 |
| 4 | 0.0 | 0.4 | 0.3 | 0.7 | 0.8 |
| 5 | 0.0 | 0.5 | 0.4 | 0.9 | 1 |
| 6 | 0.0 | 0.5 | 0.6 | 1.1 | 1.1 |
| 7 | 0.0 | 0.6 | 0.6 | 1.2 | 1.3 |
| 21.5 | 0.0 | 1.4 | 1.7 | 3.1 | 3.5 |
| 22.5 | 0.0 | 1.4 | 1.8 | 3.2 | 3.6 |
| 23.5 | 0.0 | 1.4 | 1.9 | 3.3 | 3.8 |

The data indicate that both conductive composite wicks with e-Reeds and carbon fibers and conductive composite wicks with sintered porous plastic wick and carbon fibers showed electrical energy dependent oil based fragrance vaporization capability. Higher amounts of electrical energy evaporated more fragrance into the environment.

### Example 11

### Conductive Composite Wick for Oil Based Insecticide Delivery

Four e-Reeds (e-Reeds X21193, Porex, Fairburn, Ga.) and two porous plastic rods (X 5531 UHMWPE, Porex, Fairburn, Ga.) were employed, with and without carbon fiber tow. The e-Reeds with carbon fiber conductive elements were the same as disclosed in example 6. The porous plastic rods with carbon fiber conductive elements were the same as disclosed in example 7. The rods were placed in a 50 ml Corning Conical-bottom disposable plastic tube containing liquid. One end of the e-Reeds and one end of the porous plastic rod was immersed in the liquid and the other end of each rod was outside the tube. The wicks were placed into the tube as disclosed in examples 6 and 7.

The tube was filled with Tiki BiteFighter® torch fuel (Lamplight Farms Inc. Menomonee Falls, Wis., purchased from The Home Depot) with cedar oil and mineral oil at about the 25 ml mark line. The total weight of the tube, liquid and sintered porous rod wick was recorded. The wick was connected to a DC power supply (EXTECH Digital single output DC power supply) and 9.0 V electric potential was applied to the two open ends of the sintered porous plastic wick. No voltage was applied to the e-Reeds and porous plastic rods without carbon fiber tow. A tube with Tiki BiteFighter® alone was used as control. The total weight was recorded at different times. The weight difference between the original weight and recorded weight was the amount of liquid vaporized through the system. Table 13 lists the weight loss data for the control, four e-Reed (Porex X 21193), two porous plastic rod (Porex X 5531), conductive composite wick comprising of four e-Reeds (Porex X 21193) with carbon fiber and conductive composite wick comprising two porous plastic rods (Porex X 5531) and carbon fiber.

TABLE 13
Weight loss data for the control, four e-Reeds (Porex X 21193), two
porous plastic rods (Porex X 5531), conductive composite wick
comprising four e-Reeds (Porex X 21193) with carbon fiber and
conductive composite wick comprising two porous
plastic rods (Porex X 5531) and carbon fiber over time.

| | Delivery Amount (gram) | | | | |
|---|---|---|---|---|---|
| | | | | Four | Two porous |
| | | | | e-Reeds with | plastic rods |
| Time | | Four | Two porous | carbon fiber | with carbon |
| (hrs) | Control | e-Reeds | plastic rods | 9 V | fiber 9 V |
| 0 | 0.0 | 0 | 0 | 0 | 0 |
| 1 | 0.0 | 0 | 0 | 0.1 | 0.1 |
| 2 | 0.0 | 0 | 0 | 0.2 | 0.2 |
| 3 | 0.0 | 0 | 0 | 0.2 | 0.3 |
| 4 | 0.0 | 0 | 0 | 0.3 | 0.4 |

The data indicate that both conductive composite wicks with e-Reeds and carbon fibers, and conductive composite wick with sintered porous plastic wick and carbon fibers delivered low vapor pressure Tiki BiteFighter® into the environment with 9V electricity and e-Reed and sintered porous plastic alone did not vaporize Tiki BiteFighter®.

### Example 12

### Battery Powered Fragrance Delivery Device

A three-inch long conductive composite wick as described in the example 5 was used. The conductive composite wick comprised bicomponent fiber, conductive carbon fiber, and colored monocomponent fiber. The carbon fiber conductive element was embedded in the center of conductive composite fiber wick.

The carbon fiber element inside the wick had an electrical resistance of 4 ohms. The two ends of the carbon fiber inside the wick were connected to a power source containing two AA batteries connected sequentially. The wick was inserted into a SC Johnson Glade® Plugins® refill with sweet pea and lilac fragrance (SC Johnson, Racine, Wis.). Three individuals in a 100 square feet room reported their sensation of the fragrance before and after the power was switched on. These individuals reported no sensation of fragrance before the power was turned on. They reported a significant increase in their perception of the fragrance after the power was on for two minutes.

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention.

1. A conductive composite wick comprising a porous wicking element and a conductive element, wherein the porous wicking element is coupled to the conductive element.

2. The conductive composite wick of claim 1, wherein the porous wicking element comprises a fiber or a sintered porous plastic.

3. The conductive composite wick of claim 1, wherein the conductive element comprises carbon, metal or a metal alloy.

4. The porous wicking element of claim 2, wherein the fiber comprises a staple fiber, a continuous fiber, a bicomponent fiber, a mono-component fiber or a combination thereof.

5. The porous wicking element of claim 2, wherein the sintered porous plastic comprises polyethylene or polypropylene.

6. The porous wicking element of claim 5, wherein the polyethylene is high density polyethylene, very high molecular weight polyethylene or ultrahigh molecular weight polyethylene.

7. The fiber porous wicking element of claim 2, comprising a pore size from about 10 microns to about 200 microns and an average pore size volume from about 40% to about 95%.

8. The sintered porous plastic wicking element of claim 2, comprising a pore size from about 10 microns to about 200 microns and an average pore size volume from about 10% to about 70%.

9. A device comprising:
a conductive composite wick comprising a porous wicking element and a conductive element, wherein the porous wicking element is coupled to the conductive element; and,
an energy source connected to the conductive element.

10. The device of claim 9, wherein the energy source comprises a heating element or an electrical power source.

11. The device of claim 9, further comprising a container for housing the device, the container having an opening.

12. The device of claim 9, further comprising a reservoir comprising a vaporizable material.

13. A method for releasing a vaporizable material into the air comprising:
providing a device comprising:
a container with an opening and containing a reservoir of vaporizable material;
a conductive composite wick comprising a porous wicking element and a conductive element, wherein the porous wicking element is coupled to the conductive element, a first portion of the porous wicking element contacts the vaporizable material in the reservoir, and a second portion of the porous wicking element is located outside the reservoir of the vaporizable material; and,
an energy source connected to the conductive element;
applying energy from the energy source to the conductive element;
wicking the vaporizable material from the reservoir through the porous wicking element; and,
releasing the vaporizable material from the porous wicking element.

14. The energy source of claim 9, wherein the energy source comprises a DC energy source or an AC energy source.

15. The conductive element of claim 1, wherein the conductive element is thermally or electrically conductive.

16. The vaporizable material of claim 12, wherein the vaporizable material comprises a fragrance, an insecticide, an insect repellant, a disinfectant, a deodorant, a pheromone, a pharmaceutical agent or a combination thereof.

17. The vaporizable material of claim 13, wherein the vaporizable material comprises a fragrance, an insecticide, an insect repellant, a disinfectant, a deodorant, a pheromone, a pharmaceutical agent or a combination thereof.

18. The conductive element of claim 9, wherein the conductive element is thermally or electrically conductive.

19. The conductive element of claim 13, wherein the conductive element is thermally or electrically conductive.

20. The energy source of claim 13, wherein the energy source comprises a DC energy source or an AC energy source.
